SEQUENCE LISTING (3 sequences)

SEQ ID NO: 1 - protein, 1273 aa, Sars-Cov-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 685 aa, Sars-Cov-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 598 aa, Human
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

(19) United States
(12) Patent Application Publication
(10) Pub. No.: US 2023/0168245 A1
(43) Pub. Date: Jun. 1, 2023

(54) Lateral Flow Device for Detection of Neutralizing Antibodies Against SARS-COV-2

(71) Applicant: Sorrento Therapeutics, Inc., San Diego, CA (US)

(72) Inventor: Henry Hongjun Ji, Rancho Santa Fe, CA (US)

(73) Assignee: Sorrento Therapeutics, Inc., San Diego, CA (US)

(21) Appl. No.: 17/920,772

(22) PCT Filed: Apr. 23, 2021
(86) PCT No.: PCT/US2021/028892

§ 371 Date: Oct. 21, 2022

Related U.S. Application Data
(60) Provisional application No. 63/014,962, filed on Apr. 24, 2020.
(60) Provisional application No. 63/041,645, filed on Jun. 19, 2020.

(51) Int. Cl. G01N 33/543; G01N 33/569

(52) U.S. Cl. CPC G01N 33/54386; G01N 33/56983; G01N 2333/165; G01N 2469/20

(57) ABSTRACT

The present disclosure provides a lateral flow device, and methods of use of the device, for accurately and rapidly detecting a COVID-19 infection in a subject. The lateral flow device described herein detects in a liquid sample from the subject the presence or absence of neutralizing and non-neutralizing anti-S1 spike antibodies as IgM and IgG types. The anti-S1 antibodies neutralizing antibodies (e.g., both IgM and IgG) can block binding between ACE2 antigen and S1 spike protein which is the basis for the design of the lateral flow device.

### RELATED APPLICATIONS

This application claims priority to U.S. Provisional Patent Application No. 63/014,962, filed on Apr. 24, 2020, and to U.S. Provisional Patent Application No. 63/041,645, filed Jun. 19, 2020, the entire contents of each of which are expressly incorporated herein by reference.

Throughout this application various publications, patents, and/or patent applications are referenced. The disclosures of the publications, patents and/or patent applications are hereby incorporated by reference in their entireties into this application in order to more fully describe the state of the art to which this disclosure pertains. To the extent any material incorporated by reference conflicts with the express content of this application, the express content controls.

### INTRODUCTION AND SUMMARY

The present disclosure provides a lateral flow device, and methods of use of the device, for accurately and rapidly detecting a COVID-19 infection in a subject. The lateral flow device described herein detects the presence of IgG anti-S1 spike antibodies, IgM anti-S1 antibodies, and neutralizing antibodies (e.g., both IgM and IgG) that block binding between ACE2 antigen and S1 spike protein.

The disclosure includes the following embodiments. Embodiment 1 is a lateral flow device comprising: a plurality of lateral flow regions arranged in the order:

a) a sample application zone (110) for dispensing a liquid sample thereupon wherein the sample application zone (110) comprises an absorbent material;
b) a conjugate pad (140) comprising an absorbent material and a plurality of pre-set gold-conjugates which include (i) a plurality of coronavirus S1 spike polypeptide-gold conjugates and (ii) a plurality of biotin-gold conjugates;
c) a detection zone (150) comprising a lateral flow membrane (160) comprising an absorbent material which includes a plurality of test lines and a control line, wherein the plurality of test lines includes (i) a test line (170a) comprising a plurality of pre-set immobilized human ACE2 receptor polypeptide capture reagents, (ii) a test line (170b) comprising a plurality of pre-set immobilized anti-human IgM capture antibodies, (iii) a test line (170c) comprising a plurality of pre-set immobilized anti-human IgG capture antibodies, and the control line (180) comprises a plurality of pre-set immobilized capture reagents that bind biotin; and
d) an absorbent pad (190) comprising a second absorbent material, wherein the lateral flow membrane (160) is in fluid communication with the absorbent pad (190) and wherein (i) the sample application zone (110) is in fluid communication with the conjugate pad (140) and the conjugate pad (140) is in fluid communication with the lateral flow membrane (160); or (ii) the sample pad (130) is in fluid communication with the lateral flow membrane (160).

Embodiment 2 is a kit comprising a detectably labeled S1 spike conjugate, a detectably labeled biotin conjugate, and a lateral flow device, wherein the lateral flow device comprises a plurality of lateral flow regions arranged in the order:

a) a sample application zone (110) for dispensing a liquid sample thereupon wherein the sample application zone (110) comprises an absorbent material;
b) at least one of (i) a conjugate pad (140) comprising an absorbent material and a plurality of pre-set detectably labeled conjugates which include (i) a plurality of detectably labeled coronavirus S1 spike polypeptide conjugates and (ii) a plurality of detectably labeled biotin conjugates or (ii) a sample pad (130);
c) a detection zone (150) comprising a lateral flow membrane (160) comprising an absorbent material which includes a plurality of test lines and a control line, wherein the plurality of test lines includes (i) a test line (170a) comprising a plurality of pre-set immobilized human ACE2 receptor polypeptide capture reagents, (ii) a test line (170b) comprising a plurality of pre-set immobilized anti-human IgM capture antibodies, (iii) a test line (170c) comprising a plurality of pre-set immobilized anti-human IgG capture antibodies, and the control line (180) comprises a plurality of pre-set immobilized capture reagents that bind biotin; and
d) an absorbent pad (190) comprising a second absorbent material, wherein the lateral flow membrane (160) is in fluid communication with the absorbent pad (190) and wherein (i) the sample application zone (110) is in fluid communication with the conjugate pad (140) and the conjugate pad (140) is in fluid communication with the lateral flow membrane (160); or (ii) the sample pad (130) is in fluid communication with the lateral flow membrane (160).

Embodiment 3 is the lateral flow device of embodiment 1 or the kit of embodiment 2, wherein the detectably labeled coronavirus S1 spike polypeptide conjugates comprise coronavirus S1 spike polypeptide gold-conjugates.

Embodiment 4 is the lateral flow device or kit of any one of the preceding embodiments, wherein the detectably labeled biotin conjugates comprise biotin gold-conjugates.

Embodiment 5 is the lateral flow device or kit of any one of the preceding embodiments, wherein the test line (170a) comprising the plurality of pre-set immobilized human ACE2 receptor polypeptide capture reagents is the first test line, the test line (170b) comprising the plurality of pre-set immobilized anti-human IgM capture antibodies is the second test line, and the test line (170c) comprising the plurality of pre-set immobilized anti-human IgG capture antibodies is the third test line, in order of increasing distance from the sample application zone.

Embodiment 6 is the lateral flow device or kit of any one of the preceding embodiments, wherein the sample application zone (110) comprises a sample pad (130) and an optional sample port (120), wherein the sample port (120) if present is in fluid communication with the sample pad (130).

Embodiment 7 is the lateral flow device or kit of any one of the preceding embodiments, wherein the coronavirus S1 spike polypeptide in the plurality of detectably labeled coronavirus S1 spike polypeptide conjugates comprises S1 spike polypeptide from SARS-CoV-2 and having the amino acid sequence of SEQ ID NO:2.

Embodiment 8 is the lateral flow device or kit of any one of the preceding embodiments, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents at the test line (170a).

Embodiment 9 is the lateral flow device or kit of any one of the preceding embodiments, wherein the coronavirus S1 spike polypeptide in the plurality of detectably labeled coronavirus S1 spike polypeptide conjugates comprises a receptor binding domain (RBD) that binds a human anti-S1 neutralizing IgM antibody, or the receptor binding domain (RBD) binds a human anti-S1 neutralizing IgG antibody.

Embodiment 10 is the lateral flow device or kit of any one of the preceding embodiments, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents, and the receptor binding domain (RBD) binds a human anti-S1 neutralizing IgM antibody.

Embodiment 11 is the lateral flow device or kit of embodiment 10, wherein binding between the receptor binding domain (RBD) of the coronavirus S1 spike polypeptide and the human anti-S1 neutralizing IgM antibody blocks binding between the receptor binding domain (RBD) of the coronavirus S1 spike polypeptide and the pre-set immobilized human ACE2 receptor polypeptide capture reagents at the test line (170a).

Embodiment 12 is the lateral flow device or kit of any one of the preceding embodiments, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents, and the receptor binding domain (RBD) binds a human anti-S1 neutralizing IgG antibody.

Embodiment 13 is the lateral flow device or kit of embodiment 12, wherein binding between the receptor binding domain (RBD) of the coronavirus S1 spike polypeptide and the human anti-S1 neutralizing IgG antibody blocks binding between the receptor binding domain (RBD) of the coronavirus S1 spike polypeptide and the pre-set immobilized human ACE2 receptor polypeptide capture reagents at the test line (170a).

Embodiment 14 is the lateral flow device or kit of any one of the preceding embodiments, wherein the coronavirus S1 spike polypeptide in the plurality of detectably labeled coronavirus S1 spike polypeptide conjugates comprises non-receptor binding domains that bind a human anti-S1 non-neutralizing IgM antibody.

Embodiment 15 is the lateral flow device or kit of embodiment 14, wherein binding between the non-receptor binding domain of the coronavirus S1 spike polypeptide and the human anti-S1 non-neutralizing IgM antibody does not block binding between the receptor binding domain (RBD) of the coronavirus S1 spike polypeptide and the pre-set immobilized human ACE2 receptor polypeptide capture reagents at the test line (170a).

Embodiment 16 is the lateral flow device or kit of any one of the preceding embodiments, wherein the coronavirus S1 spike polypeptide in the plurality of detectably labeled coronavirus S1 spike polypeptide conjugates comprises non-receptor binding domains that bind a human anti-S1 non-neutralizing IgG antibody.

Embodiment 17 is the lateral flow device or kit of embodiment 16, wherein binding between the non-receptor binding domain of the coronavirus S1 spike polypeptide and the human anti-S1 non-neutralizing IgG antibody does not block binding between the receptor binding domain (RBD) of the coronavirus S1 spike polypeptide and the pre-set immobilized human ACE2 receptor polypeptide capture reagents at the test line (170a).

Embodiment 18 is the lateral flow device or kit of any one of the preceding embodiments, wherein the plurality of detectably labeled coronavirus S1 spike polypeptide conjugates comprise gold nanoparticles conjugated to coronavirus S1 spike polypeptides, and wherein the gold nanoparticles have a detectable color.

Embodiment 19 is the lateral flow device or kit of any one of the preceding embodiments, wherein the plurality of detectably labeled biotin conjugates comprise gold nanoparticles conjugated to biotin, and wherein the gold nanoparticles have a detectable color.

Embodiment 20 is the lateral flow device or kit of any one of the preceding embodiments, wherein the pre-set immobilized human ACE2 receptor polypeptide capture reagents in the test line (170a) comprise an epitope that binds a receptor binding domain (RBD) of an S1 spike polypeptide from SARS-CoV-2, wherein the pre-set immobilized human ACE2 receptor polypeptide comprises the amino acid sequence of SEQ ID NO:3, or a portion thereof.

Embodiment 21 is the lateral flow device or kit of any one of the preceding embodiments, wherein the pre-set immobilized anti-human IgM capture antibodies in the test line (170b) bind a human anti-S1 IgM antibody (e.g., neutralizing and non-neutralizing antibodies).

Embodiment 22 is the lateral flow device or kit of embodiment 21, wherein the pre-set immobilized anti-human IgM capture antibodies in the test line (170b) bind a plurality of a complex comprising the human anti-S1 IgM antibody bound to the detectably labeled coronavirus S1 spike polypeptide conjugate.

Embodiment 23 is the lateral flow device or kit of any one of the preceding embodiments, wherein the pre-set immobilized anti-human IgG capture antibodies in the third test line (170b) bind a human anti-S1 IgG antibody (e.g., neutralizing and non-neutralizing antibodies).

Embodiment 24 is the lateral flow device or kit of embodiment 23, wherein the pre-set immobilized anti-human IgG capture antibodies in the third test line (170c) bind a plurality of a complex comprising the human anti-S1 IgG antibody bound to the detectably labeled coronavirus S1 spike polypeptide conjugate.

Embodiment 25 is the lateral flow device or kit of any one of the preceding embodiments, wherein the pre-set immobilized capture reagents that bind biotin at the control line (180) bind a plurality of detectably labeled biotin conjugates.

Embodiment 26 is the lateral flow device or kit of any one of the preceding embodiments, which is disposed in a housing which includes a base, a lid, two end walls and two side walls.

Embodiment 27 is the lateral flow device or kit of embodiment 26, wherein the lid of the housing includes a first cut-out region at the position of the sample port (120) or the sample pad (130) for liquid sample dispensing, and the lid includes a second cut-out region at the detection zone (150) for use as an observation window.

Embodiment 28 is the lateral flow device or kit of any one of the preceding embodiments, wherein the capture reagents that bind biotin comprise avidin, streptavidin, NEUTRAVIDIN, EXTRAVIDIN, CAPTAVIDIN, or NEUTRALITE AVIDIN, or a truncated form thereof that retains biotin-binding activity, optionally wherein the avidin, streptavidin, NEUTRAVIDIN, EXTRAVIDIN, CAPTAVIDIN, or NEUTRALITE AVIDIN is glycosylated.

Embodiment 29 is the lateral flow device or kit of any one of the preceding embodiments, wherein the lateral flow device further comprises a second plurality of lateral flow regions arranged in the order:

2a) a second sample application zone for dispensing a liquid sample thereupon wherein the sample application zone comprises an absorbent material;
2b) at least one of (i) a second conjugate pad comprising an absorbent material and a plurality of pre-set detectably labeled conjugates which include (1) a plurality of detectably labeled coronavirus S1 spike polypeptide conjugates and (2) a plurality of detectably labeled biotin conjugates, or (ii) a second sample pad;
2c) a second detection zone comprising a lateral flow membrane comprising an absorbent material which includes a test line comprising a plurality of pre-set immobilized human ACE2 receptor polypeptide capture reagents and a control line comprising a plurality of pre-set immobilized capture reagents that bind biotin; and
2d) a second absorbent pad comprising an absorbent material,

wherein the lateral flow membrane is in fluid communication with the absorbent pad, and wherein (i) the sample application zone is in fluid communication with the second conjugate pad and the second conjugate pad is in fluid communication with the lateral flow membrane of the second detection zone, or (ii) the second sample pad (130) is in fluid communication with the lateral flow membrane of the second detection zone.

Embodiment 30 is the kit of any one of embodiments 2-29, wherein the lateral flow device further comprises a second plurality of lateral flow regions arranged in the order:

2a) a second sample application zone for dispensing a liquid sample thereupon wherein the sample application zone comprises an absorbent material;
2b) at least one of (i) a second conjugate pad comprising an absorbent material and a plurality of pre-set detectably labeled conjugates which include (1) a plurality of detectably labeled coronavirus S1 spike polypeptide conjugates and (2) a plurality of detectably labeled biotin conjugates, or (ii) a second sample pad;
2c) a second detection zone comprising a lateral flow membrane comprising an absorbent material which includes a test line comprising a plurality of pre-set immobilized human ACE2 receptor polypeptide capture reagents and a control line comprising a plurality of pre-set immobilized capture reagents that bind biotin; and
2d) a second absorbent pad comprising an absorbent material,

wherein the lateral flow membrane is in fluid communication with the absorbent pad, and wherein (i) the sample application zone is in fluid communication with the second conjugate pad and the second conjugate pad is in fluid communication with the lateral flow membrane of the second detection zone, or (ii) the second sample pad (130) is in fluid communication with the lateral flow membrane of the second detection zone.

Embodiment 31 is the lateral flow device or kit of embodiment 29 or the kit of embodiment 30, wherein the second sample application zone comprises the second sample pad and an optional sample port, wherein the optional sample port if present is in fluid communication with the second sample pad.

Embodiment 32 is the lateral flow device or kit of any one of embodiments 29-31, wherein the detectably labeled coronavirus S1 spike polypeptide conjugates in the second conjugate pad comprise S1 spike polypeptide from SARS-CoV-2 and having the amino acid sequence of SEQ ID NO:2.

Embodiment 33 is the lateral flow device or kit of any one of embodiments 29-32, wherein the detectably labeled coronavirus S1 spike polypeptide conjugates in the second conjugate pad comprise a receptor binding domain (RBD) that binds an epitope on the pre-set immobilized human ACE2 receptor polypeptide capture reagents at the test line (170d).

Embodiment 34 is the lateral flow device or kit of any one of embodiments 29-33, wherein the detectably labeled coronavirus S1 spike polypeptide conjugates in the second conjugate pad comprise a receptor binding domain (RBD) that binds a human anti-S1 neutralizing IgM antibody, or the receptor binding domain (RBD) binds a human anti-S1 neutralizing IgG antibody.

Embodiment 35 is the lateral flow device or kit of any one of embodiments 29-34, wherein the detectably labeled coronavirus S1 spike polypeptide conjugates in the second conjugate pad comprise a receptor binding domain (RBD) that binds an epitope on the pre-set immobilized human ACE2 receptor polypeptide capture reagents at the test line (170d), and the receptor binding domain (RBD) binds a human anti-S1 neutralizing IgM antibody.

Embodiment 36 is the lateral flow device or kit of any one of embodiments 29-35, wherein the coronavirus S1 spike polypeptide in the plurality of detectably labeled coronavirus S1 spike polypeptide conjugates of the second plurality of lateral flow regions comprises a receptor binding domain (RBD) that binds an epitope on the pre-set immobilized human ACE2 receptor polypeptide capture reagents at the test line (170d), and the receptor binding domain (RBD) binds a human anti-S1 neutralizing IgG antibody.

Embodiment 37 is the lateral flow device or kit of any one of embodiments 29-36, wherein the detectably labeled coronavirus S1 spike polypeptide conjugates in the second conjugate pad comprise non-receptor binding domains that bind a human anti-S1 non-neutralizing IgM antibody.

Embodiment 38 is the lateral flow device or kit of embodiment 37, wherein binding between the non-receptor binding domain of the coronavirus S1 spike polypeptide and the human anti-S1 non-neutralizing IgM antibody does not block binding between the receptor binding domain (RBD) of the coronavirus S1 spike polypeptide and the pre-set immobilized human ACE2 receptor polypeptide capture reagents at the test line.

Embodiment 39 is the lateral flow device or kit of any one of embodiments 29-38, wherein the detectably labeled coronavirus S1 spike polypeptide conjugates in the second conjugate pad comprise non-receptor binding domains that bind a human anti-S1 non-neutralizing IgG antibody.

Embodiment 40 is the lateral flow device or kit of any one of embodiments 29-39, wherein the detectably labeled coronavirus S1 spike polypeptide conjugates comprise coronavirus S1 spike polypeptide gold-conjugates.

Embodiment 41 is the lateral flow device or kit of any one of embodiments 29-40, wherein the detectably labeled biotin conjugates in the second conjugate pad comprise biotin-gold conjugates.

Embodiment 42 is the lateral flow device or kit of the immediately preceding embodiment, wherein the biotin-gold conjugates comprise gold nanoparticles conjugated to biotin, and wherein the gold nanoparticles have a detectable color.

Embodiment 43 is the lateral flow device or kit of any one of embodiments 29-42, wherein the pre-set immobilized human ACE2 receptor polypeptide capture reagents in the test line of the second detection zone comprise an epitope that binds a receptor binding domain (RBD) of an S1 spike polypeptide from SARS-CoV-2, wherein the pre-set immobilized human ACE2 receptor polypeptide comprises the amino acid sequence of SEQ ID NO:3, or a portion thereof.

Embodiment 44 is the lateral flow device or kit of any one of embodiments 29-43, which is disposed in a housing which includes a base, a lid, two end walls and two side walls, wherein the lid of the housing includes a third cut-out region at the position of the sample port or the sample pad for liquid sample dispensing, and the lid includes a fourth cut-out region at the detection zone for use as an observation window,

wherein the third and fourth cut-out regions are positioned over the second plurality of lateral flow regions.

Embodiment 45 is a method for detecting in a liquid sample from a subject the presence or absence of anti-S1 antibody analytes of IgM and/or IgG type, the method comprising the steps of:

a) providing a liquid sample from the subject, wherein the liquid sample is suspected of containing human anti-S1 non-neutralizing antibody analytes of IgM and/or IgG type, and wherein the liquid sample is suspected of containing human anti-S1 neutralizing antibody analytes of IgM and/or IgG type;
b) dispensing the liquid sample onto the sample application zone (110) of a lateral flow device of any one of embodiments 1-44 (i) under a condition that is suitable for lateral flow of the liquid sample and the human anti-S1 non-neutralizing and neutralizing antibody analytes that may be contained therein, wherein the lateral flow moves the liquid sample or aliquot and the human anti-S1 non-neutralizing and neutralizing antibody analytes, if present, from the sample application zone (110) through the conjugate pad (140) through the detection zone (150) and through the absorbent pad (190), and (ii) under a condition that is suitable for lateral flow of the pre-set detectably labeled coronavirus S1 spike polypeptide conjugates and the pre-set detectably labeled biotin conjugates from the conjugate pad (140) through the detection zone (150) and through the absorbent pad (190), and (iii) under a condition that is suitable for binding of flowing detectably labeled coronavirus S1 spike polypeptide conjugates to the human anti-S1 non-neutralizing antibody analytes (if present) to form at least one S1 polypeptide-non-neutralizing antibody complex, and suitable for binding of flowing detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex, and (iv) under a condition that is suitable for binding flowing detectably labeled coronavirus S1 spike polypeptide conjugates to the pre-set immobilized human ACE2 receptor polypeptide capture reagents, and is suitable for binding the detectably labeled S1 polypeptide-non-neutralizing antibody complex to the pre-set immobilized anti-human IgM and IgG capture antibodies, and is suitable for binding the S1 polypeptide-neutralizing antibody complex to the pre-set immobilized anti-human IgM and IgG capture antibodies, and is suitable for binding flowing detectably labeled biotin conjugates to the pre-set immobilized capture reagents that bind biotin;
c) waiting a sufficient time for a color change to develop at the test line (170a), the test line (170b), the test line (170c) and the control line (180), or permitting a color change to develop at one or more test and/or control lines; and
d) detecting the color change at the test line (170a), the test line (170b), the test line (170c) and/or the control line (180).

Embodiment 46 is a method for detecting in a liquid sample from a subject the presence or absence of anti-S1 antibody analytes of IgM and/or IgG type, the method comprising the steps of:

a) providing a liquid sample from the subject, wherein the liquid sample is suspected of containing human anti-S1 non-neutralizing antibody analytes of IgM and/or IgG type, and wherein the liquid sample is suspected of containing human anti-S1 neutralizing antibody analytes of IgM and/or IgG type, and mixing the liquid sample with a detectably labeled S1 spike conjugate and a detectably labeled biotin conjugate;
b) dispensing the liquid sample onto the sample application pad (130) or the sample port (120) of a lateral flow device of the kit of any one of embodiments 2-44 (i) under a condition that is suitable for lateral flow of the liquid sample and the human anti-S1 non-neutralizing and neutralizing antibody analytes that may be contained therein, wherein the lateral flow moves the liquid sample or aliquot and the human anti-S1 non-neutralizing and neutralizing antibody analytes, if present, from the sample application pad (130) or the sample port (120) through the detection zone (150) and through the absorbent pad (190), and (ii) under a condition that is suitable for lateral flow of the detectably labeled coronavirus S1 spike polypeptide conjugates and the detectably labeled biotin conjugates from the conjugate pad (140) through the detection zone (150) and through the absorbent pad (190), and (iii) under a condition that is suitable for binding of the detectably labeled coronavirus S1 spike polypeptide conjugates to the human anti-S1 non-neutralizing antibody analytes (if present) to form at least one S1 polypeptide-non-neutralizing antibody complex, and suitable for binding of the detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex, and (iv) under a condition that is suitable for binding flowing detectably labeled coronavirus S1 spike polypeptide conjugates to the pre-set immobilized human ACE2 receptor polypeptide capture reagents, and is suitable for binding the detectably labeled S1 polypeptide-non-neutralizing antibody complex to the pre-set immobilized anti-human IgM and IgG capture antibodies, and is suitable for binding the S1 polypeptide-neutralizing antibody complex to the pre-set immobilized anti-human IgM and IgG capture antibodies, and is suitable for binding flowing detectably labeled biotin conjugates to the pre-set immobilized capture reagents that bind biotin;
c) waiting a sufficient time for a color change to develop at the test line (170a), the test line (170b), the test line (170c) and the control line (180), or permitting a color change to develop at one or more test and/or control lines; and
d) detecting the color change at the test line (170a), the test line (170b), the test line (170c) and/or the control line (180).

Embodiment 47 is a method for detecting in a liquid sample from a subject the presence or absence of anti-S1 antibody analytes of IgM and/or IgG type, the method comprising the steps of:

a) providing a first liquid sample from the subject and a second liquid sample of known composition, wherein the first liquid sample is suspected of containing human anti-S1 non-neutralizing antibody analytes of IgM and/or IgG type, and wherein the first liquid sample is suspected of containing human anti-S1 neutralizing antibody analytes of IgM and/or IgG type;
b) dispensing the first liquid sample onto the sample application zone and dispensing the second liquid sample onto the second sample application zone of the lateral flow device of any one of embodiments 29-44 (i) under a condition that is suitable for lateral flow of the liquid samples, wherein the lateral flow moves the first liquid sample from the sample application zone through the conjugate pad, through the detection zone, and through the absorbent pad, and moves the second liquid sample from the second sample application zone through the second conjugate pad, through the second detection zone, and through the second absorbent pad, and (ii) under a condition that is suitable for lateral flow of the pre-set detectably labeled coronavirus S1 spike polypeptide conjugates and the pre-set detectably labeled biotin conjugates from the conjugate pad and second conjugate pad through the detection zone and second detection zone and through the absorbent pad and second absorbent pad, and (iii) under a condition that is suitable for binding of flowing detectably labeled coronavirus S1 spike polypeptide conjugates to the human anti-S1 non-neutralizing antibody analytes (if present) to form at least one S1 polypeptide-non-neutralizing antibody complex, and suitable for binding of flowing detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex and that is suitable for binding flowing detectably labeled coronavirus S1 spike polypeptide conjugates to the pre-set immobilized human ACE2 receptor polypeptide capture reagents at the test line of the second detection zone, and is suitable for binding flowing detectably labeled biotin conjugates to the pre-set immobilized capture reagents that bind biotin at the control line of the second detection zone;
c) waiting a sufficient time for a color change to develop at the test lines and the control lines, or permitting a color change to develop at one or more test and/or control lines; and
d) detecting the color change at one or more of the test lines and/or the control lines.

Embodiment 48 is a method for detecting in a liquid sample from a subject the presence or absence of anti-S1 antibody analytes of IgM and/or IgG type, the method comprising the steps of:

a) providing a first liquid sample from the subject and a second liquid sample of known composition, wherein the first liquid sample is suspected of containing human anti-S1 non-neutralizing antibody analytes of IgM and/or IgG type, and wherein the first liquid sample is suspected of containing human anti-S1 neutralizing antibody analytes of IgM and/or IgG type;
b) mixing the first liquid sample with a detectably labeled S1 spike conjugate and a detectably labeled biotin conjugate, and mixing the second liquid sample with a detectably labeled S1 spike conjugate and a detectably labeled biotin conjugate;
c) dispensing the first liquid sample onto the sample pad or sample port and dispensing the second liquid sample onto the second sample pad or second sample port of the lateral flow device of the kit of any one of embodiments 30-44 (i) under a condition that is suitable for lateral flow of the liquid samples, wherein the lateral flow moves the first liquid sample from the sample application zone through the detection zone, and through the absorbent pad, and moves the second liquid sample from the second sample application zone through the second detection zone, and through the second absorbent pad, and (ii) under a condition that is suitable for lateral flow of the detectably labeled coronavirus S1 spike polypeptide conjugates and the detectably labeled biotin conjugates through the detection zone and second detection zone and through the absorbent pad and second absorbent pad, and (iii) under a condition that is suitable for binding of the detectably labeled coronavirus S1 spike polypeptide conjugates to the human anti-S1 non-neutralizing antibody analytes (if present) to form at least one S1 polypeptide-non-neutralizing antibody complex, and suitable for binding of the detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex and that is suitable for binding flowing detectably labeled coronavirus S1 spike polypeptide conjugates to the pre-set immobilized human ACE2 receptor polypeptide capture reagents at the test line of the second detection zone, and is suitable for binding flowing detectably labeled biotin conjugates to the pre-set immobilized capture reagents that bind biotin at the control line of the second detection zone;
d) waiting a sufficient time for a color change to develop at the test lines and the control lines, or permitting a color change to develop at one or more test and/or control lines; and
e) detecting the color change at one or more of the test lines and/or the control lines.

Embodiment 49 is the method of embodiment 47 or 48, wherein the detecting the color change at the test line (170b) comprising the plurality of pre-set immobilized anti-human IgM capture antibodies indicates the liquid sample contains human anti-S1 spike IgM antibodies and the subject has an active SARS-Cov-2 infection and is likely to be contagious.

Embodiment 50 is the method of any one of embodiments 47-49, wherein the detecting the color change at the test line (170c) comprising the plurality of pre-set immobilized anti-human IgG capture antibodies indicates the liquid sample contains human anti-S1 spike IgG antibodies and the subject has been infected with SARS-CoV-2 in the past but may not currently be contagious.

Embodiment 51 is the method of any one of the embodiments 47-50, wherein the detecting the color change at the test line (170a) comprising the plurality of pre-set immobilized human ACE2 receptor polypeptide capture reagents indicates the liquid sample does not contain detectable human anti-S1 spike IgM neutralizing antibodies and does not contain detectable human anti-S1 spike IgG neutralizing antibodies.

Embodiment 52 is the method of any one of embodiments 47-51, wherein a lack of a color change at the test line (170a) comprising the plurality of pre-set immobilized human ACE2 receptor polypeptide capture reagents indicates the liquid sample contains detectable human anti-S1 spike IgM neutralizing antibodies and/or the liquid sample contains detectable human anti-S1 spike IgG neutralizing antibodies.

### DESCRIPTION OF THE DRAWINGS

FIG. 1 is a schematic showing a non-limiting embodiment of a side view of a lateral flow device (100). The bold arrow at the bottom of the schematic shows the lateral flow direction.

FIG. 2 is a schematic showing a non-limiting embodiment of a top view of the same lateral flow device (100) shown in FIG. 1. The bold arrow at the bottom of the schematic shows the lateral flow direction.

FIG. 3 is a schematic showing a non-limiting embodiment of a top view of a lateral flow device useful for detecting neutralizing (blocking) antibodies against S1 spike subunit from SARS-CoV-2 virus. The multiple test lines and control line (shown from bottom to top, respectively) can be detectable as different colors. The arrow indicates the direction of capillary flow.

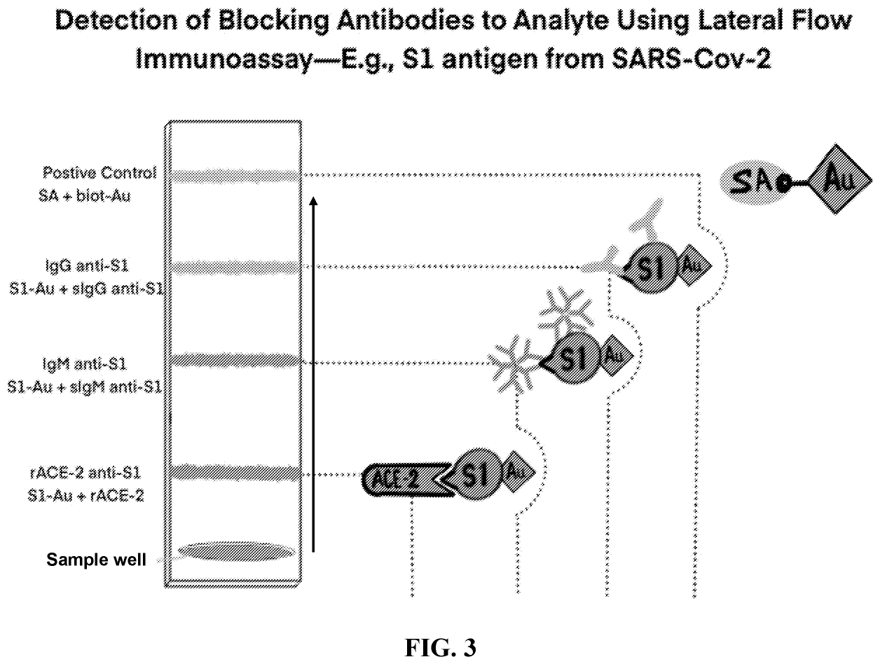

FIG. 4 is a schematic showing a three possible results with a liquid sample from a subject who may or may not have a SARS-CoV-2 infection.

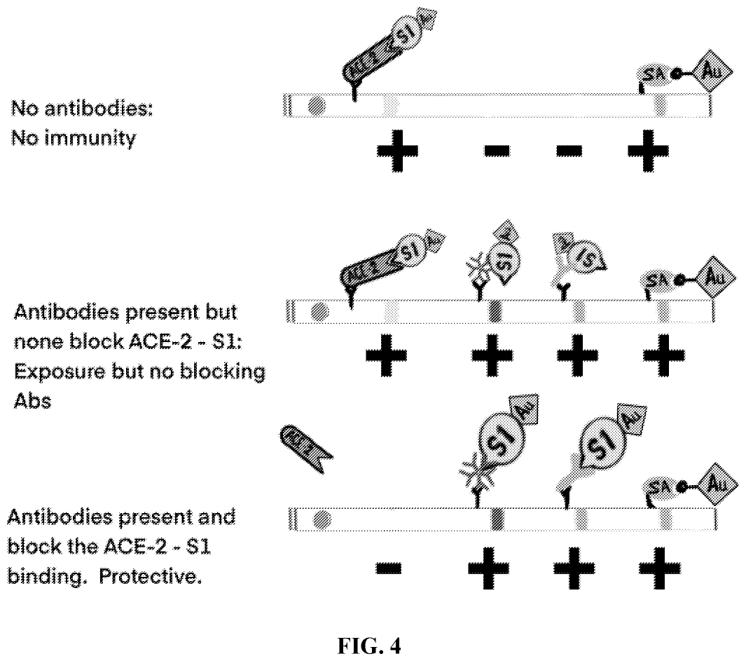

FIG. 5 is a schematic showing a portion of the lateral flow device with pre-set gold-conjugates and pre-set immobilized capture reagents. No lateral flow has occurred.

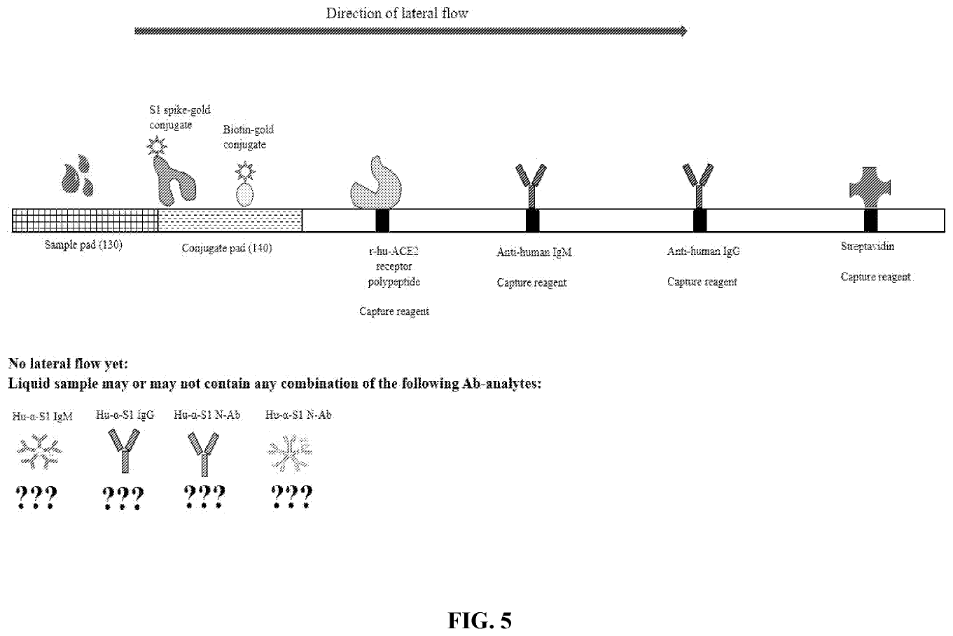

FIG. 6 is a schematic showing a portion of the lateral flow device where the dispensed liquid sample has undergone lateral flow and contains no human anti-S1 antibodies.

FIG. 7 is a schematic showing a portion of the lateral flow device where the dispensed liquid sample has undergone lateral flow and contains human anti-S1 antibodies non-neutralizing antibodies.

FIG. 8 is a schematic showing a portion of the lateral flow device where the dispensed liquid sample has undergone lateral flow and contains human anti-S1 antibodies neutralizing and non-neutralizing antibodies.

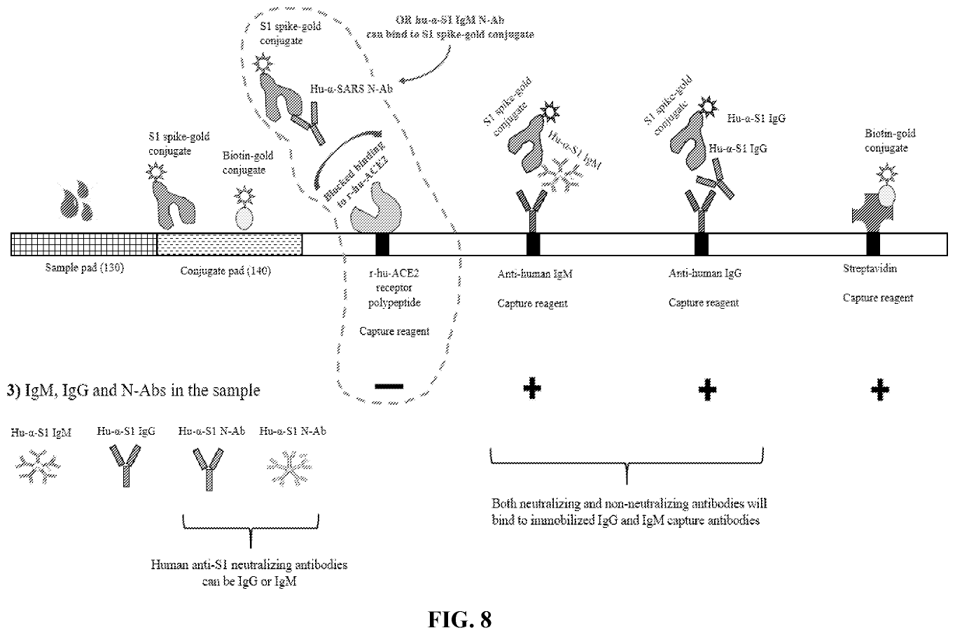

FIG. 9 shows the amino acid sequence of SARS-CoV-2 spike protein having S1 and S2 subunit sequences. The S1 subunit sequence is underlined and the S2 subunit sequence is not underlined.

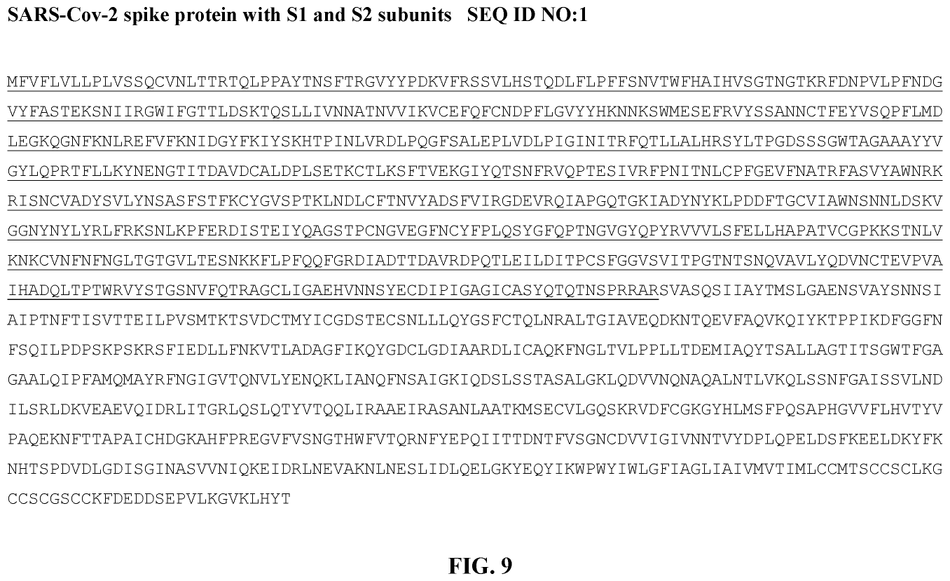

FIG. 10 shows the amino acid sequence of the S1 subunit SARS-CoV-2 sequence.

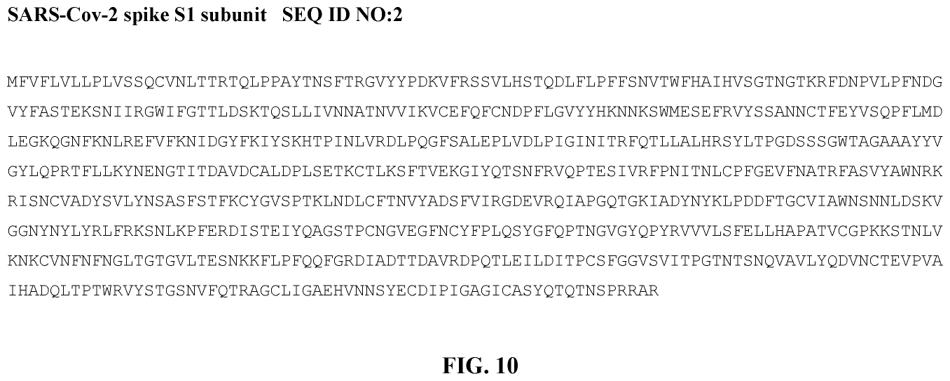

FIG. 11 shows the amino acid sequence of human ACE2 receptor polypeptide. The mutations H374N and H378N are bolded and underlined.

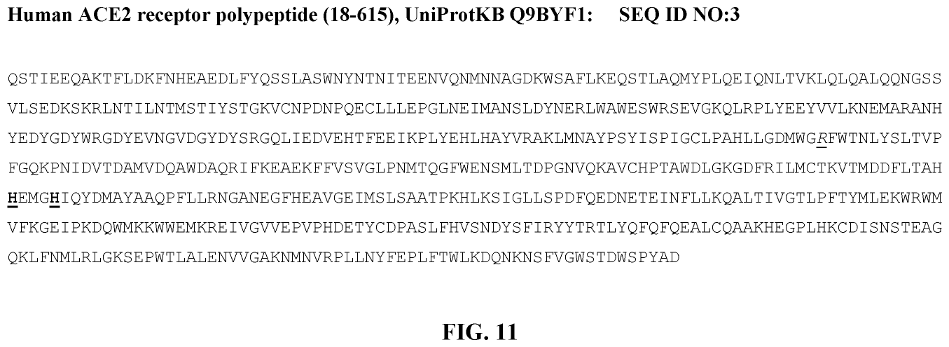

FIG. 12 illustrates a portion of an exemplary lateral flow device that comprises two test strips. The device comprises two strips. The strip on the left is a negative control strip and has a control line (C) and an ACE2 receptor polypeptide line (AC). The strip on the right is the sample test strip and has a control line (C) and test lines for IgG (G), IgM (M) and ACE2 receptor polypeptide (AC).

### DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “mutation”, “modification”, or “variation”, or related terms, refers to a change in a nucleic acid sequence or amino acid sequence that differs from a reference nucleic acid sequence or a reference amino acid sequence, respectively. Examples of mutations includes a point mutation, insertion, deletion, amino acid substitution, inversion, rearrangement, splice, sequence fusion (e.g., gene fusion or RNA fusion), truncation, transversion, translocation, non-sense mutation, sequence repeat, single nucleotide polymorphism (SNP), or other genetic rearrangement.

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)2, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations.

A “neutralizing antibody” and related terms refers to an antibody that is capable of specifically binding to the neutralizing epitope of its target antigen (e.g., coronavirus spike protein) and substantially inhibiting or eliminating the biological activity of the target antigen (e.g., coronavirus spike protein). The neutralizing antibody can reduce the biological activity of the target antigen by at least 20%, 30%, 40%, 50%, 60%, 70%, 80%, 90% or higher levels of reduced biological activity.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10′ M or less, or 10⁻⁷ M or less, or 10⁻⁸ M or less, or 10⁻⁹ M or less, or 10⁻¹⁰ M or less.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH and CH3 regions and may, or may not, include a portion of the hinge region. Two polypeptide chains each carrying a half Fc region can dimerize to form a full Fc domain. An Fc domain can bind Fc cell surface receptors and some proteins of the immune complement system. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a).

The terms “labeled,” “detectably labeled,” or related terms as used herein refers to the presence of a detectable label or moiety for detection, e.g., wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens).

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters.

In one embodiment, the amino acid sequence of a test antibody may be similar but not identical to any of the amino acid sequences of the polypeptides that make up any of the anti-S-protein antibodies, or antigen binding protein thereof, described herein. The similarities between the test antibody and the polypeptides can be at least 95%, or at or at least 96% identical, or at least 97% identical, or at least 98% identical, or at least 99% identical, to any of the polypeptides that make up any of the anti-spike protein antibodies, or antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “sample” as used herein refers to a biological sample from a negative control subject, or from a subject having or suspected of having had a coronavirus infection. Biological samples include blood, serum, plasma, whole blood, urine, nasal swab fluid, bronchoalveolar lavage (BAL) fluid, or cerebrospinal fluid (CSF). The blood sample can be obtained by fingerstick or from venous blood (whole blood, serum or plasma).

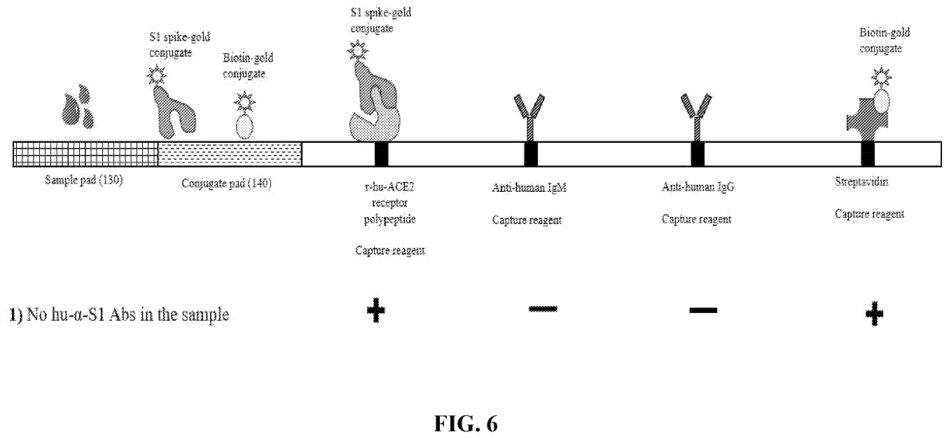

The term “S1 spike” or related terms as use herein refers to an S1 subunit of a spike protein from SARS-Cov-2 virus. In one embodiment, the S1 spike subunit comprises the amino acid sequence of SEQ ID NO:2 (see FIG. 6).

The term “fluid communication” as used herein refers to various absorbent materials described herein that are used to make a lateral flow device, where the absorbent materials are configured with each other to facilitate migration of a liquid sample in a lateral or capillary flow. The absorbent materials can be configured in end-to-end fluid connection, top-to-bottom fluid connection, or overlapping fluid connection.

Lateral Flow Device

The present disclosure provides a lateral flow device which is easy to use, requires a small volume of liquid sample from the subject to be tested, and gives visual results (e.g., colorimetric) that indicate if the subject has circulating anti-S1 spike antibodies (e.g., IgG and/or IgM antibodies) and if the circulating antibodies also include anti-S1 spike neutralizing antibodies (e.g., neutralizing IgG and/or IgM antibodies). In one embodiment, the lateral flow device gives a qualitative “yes” or “no” signal to indicate the presence or absence of circulating anti-S1 spike antibodies and neutralizing antibodies in a liquid sample from a subject suspected of having or having had an infection from a coronavirus.

The present disclosure provides a lateral flow device, and methods of use of the device, for accurately and rapidly detecting a SARS-CoV-2 infection in a subject. In some embodiments, the lateral flow device detects the presence of IgG and IgM anti-S1 spike non-neutralizing antibodies, and neutralizing antibodies that block binding between ACE2 receptor antigen and S1 spike protein from SARS-CoV-2. In some embodiments, the lateral flow device gives a color change to indicate the presence of the non-neutralizing antibodies, and gives a lack of color change to indicate the presence of the neutralizing antibodies that block binding between ACE2 receptor antigen and S1 spike protein from SARS-CoV-2. A color change at a test line indicates a positive result, while lack of a color change at a test line indicates a negative result.

In one embodiment, a positive result in a test line (e.g., test line 2) for circulating human anti-S1 spike IgM antibodies indicates that the subject has an active SARS-Cov-2 infection and is likely to be contagious. In one embodiment, a positive result in a test line (e.g., test line 3) for circulating human anti-S1 spike IgG antibodies indicates that the subject has been infected with SARS-CoV-2 in the past but may not currently be contagious. In one embodiment, a positive result in test line 1 indicates that the subject does not have circulating human anti-S1 spike neutralizing antibodies (IgM and/or IgG neutralizing antibodies), or the subject has neutralizing antibodies that are below the level of detection. In one embodiment, a negative result in test line 1 indicates that the subject has circulating human anti-S1 spike neutralizing antibodies (e.g., IgM and/or IgG neutralizing antibodies).

The results obtained from using the lateral flow device can identify subjects who have active infection and should be subjected to isolation or medical treatment. The results can also be used to identify subjects who do not have active infection and are no longer contagious. Lastly, the results from the lateral flow device can be used to identify subjects who have anti-S1 spike neutralizing antibodies and may be candidates to donate convalescent blood plasma for therapeutic use to treat other subjects having active SARS-CoV-2 infection.

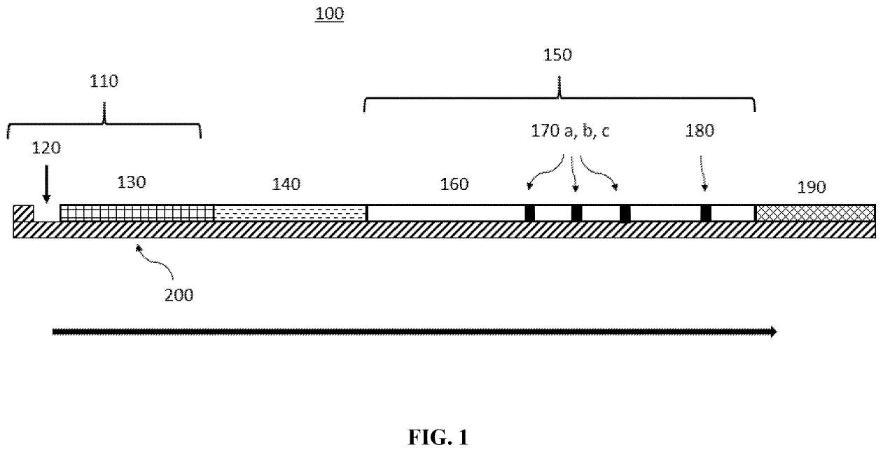

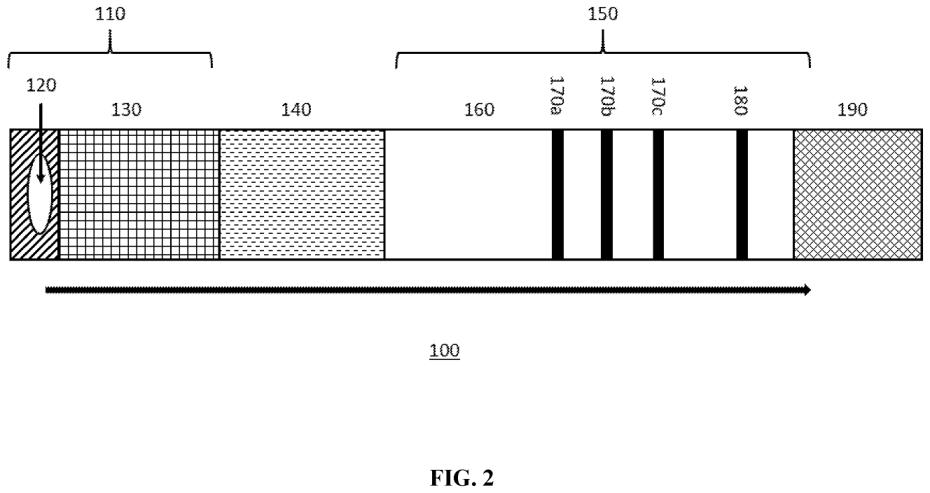

The present disclosure provides a lateral flow device (100) shown in FIGS. 1 and 2 which are illustrative and non-limiting embodiments of the disclosed subject matter. One skilled in the art will recognize that other embodiments of lateral flow devices are suitable for use in detecting a coronavirus infection in a subject.

In some embodiments, the lateral flow device comprises a self-contained, multi-layered structure having both absorbent and non-absorbent materials that form a solid-phase which is used as a device for conducting an immunoassay.

### Liquid Sample:

The liquid sample can be obtained from a human or non-human subject and is a biological liquid sample, for example blood, serum, plasma, whole blood or urine. The blood sample can be obtained by fingerstick or from venous blood (whole blood, serum or plasma). The liquid sample may be obtained from a negative control subject who has not been infected with a coronavirus, or the liquid sample may be obtained from a subject having or suspected of having had a coronavirus infection. The subject may currently be infected, or may have recently been infected, with SARS-CoV-2 coronavirus.

In a human or non-human subject who is infected with a coronavirus (e.g., SARS-CoV-2), the level of IgM antibodies in the subject increases approximately one week after infection and sometimes remain high for 2-4 months, while the level of IgG antibodies increases approximately fourteen days after infection and can be detectable up to six months or perhaps several years after the subject has recovered from the infection. Thus, the presence of anti-SARS-CoV-2 IgM and/or IgG antibodies can serve as an indicator of an active or previous coronavirus infection.

In one embodiment, the liquid sample from the human or non-human subject may contain analytes, including anti-SARS-CoV-2 antibodies, at a titer level that can be detected by the lateral flow device. The liquid sample may contain a low titer of anti-SARS-CoV-2 antibodies that is too low to detect, or may be devoid of anti-SARS-CoV-2 antibodies.

In one embodiment, the liquid sample from the human or non-human subject may contain analytes in the form of non-neutralizing and/or neutralizing antibodies of IgM and/or IgG type.

A non-neutralizing antibody is capable of binding specifically to an epitope on a S1 spike protein from SARS-Cov-2 virus. In one embodiment, the non-neutralizing antibody binds a non-receptor binding domain (non-RBD) on an S1 spike protein from SARS-CoV-2 virus. In one embodiment, the non-neutralizing antibody cannot block binding between the S1 spike protein from SARS-CoV-2 and its target receptor human ACE2. In one embodiment, the non-neutralizing antibodies present in the liquid sample comprise human anti-S1 IgM and/or human anti-S1 IgG antibodies.

A neutralizing antibody is capable of specifically binding to the neutralizing epitope of its target antigen (e.g., S1 spike protein from a SARS-CoV-2 virus) and substantially inhibiting or eliminating the biological activity of the target antigen. In one embodiment, the neutralizing antibodies that bind a SARS-CoV-2 S1 spike protein can bind an epitope on the S1 spike subunit, where the epitope on the S1 spike subunit binds a target receptor human ACE2 on a target cell. The neutralizing antibody can bind a receptor binding motif located in a receptor binding domain (RBD) of a SARS-CoV-2 S1 subunit and blocks binding between the SARS-CoV-2 S1 subunit and its target receptor human ACE2. By binding to an epitope on the SARS-CoV-2 S1 subunit, the neutralizing antibody can block attachment of coronavirus to the target cell and prevent viral entry into the target cell.

In one embodiment, the neutralizing antibodies that bind the SARS-CoV-2 S1 subunit can bind any region or any amino acid residues of the receptor binding domain (RBD) of the S1 subunit, including amino acid residues 318-565, or 318-510, or 318-510 and 565, or 426-492, or 490-510, or 460-476, or 359-362, or 331-524 (See Wanbo Tai, et al., 2020 Cellular and Molecular Immunology) (https://doi.org/10.1038/s41423-020-0400-4).

In one embodiment, the neutralizing antibodies that bind the SARS-CoV-2 S1 subunit can bind a region that overlaps with any region or any amino acid residues of the receptor binding domain (RBD) of the S1 subunit, including amino acid residues 318-565, or 318-510, or 318-510 and 565, or 426-492, or 490-510, or 460-476, or 359-362, or 331-524.

### Lateral Flow Device:

The lateral flow device may comprise a planar support (200) affixed on one side to a single type or different types of absorbent material(s) to form multiple lateral flow regions which can be arranged in the following order: an optional sample port (120); a sample pad (130); a conjugate pad (140); a lateral flow membrane (160); and an absorbent pad (190) (see FIGS. 1 and 2). In one embodiment, the support (200) is made from non-absorbent material. In one embodiment, the absorbent material(s) used to configure the lateral flow regions permits lateral or capillary flow. In one embodiment, adjacent lateral flow regions are in fluid communication with each other and can be arranged in any one or any combination of end-to-end fluid connection, top-to-bottom fluid connection, or overlapping fluid connection. In one embodiment, any of the sample port (120), sample pad (130), conjugate pad (140), lateral flow membrane (160) and/or absorbent pad (190) can be made from a material that permits lateral flow of a liquid, where the material includes nitrocellulose, nitrocellulose blends with cellulose or polyester, polyethylene membrane, nylon membrane, PVDF membrane, or paper (e.g., untreated or porous paper), rayon, glass fiber, acrylonitrile copolymer, plastic, glass or nylon.

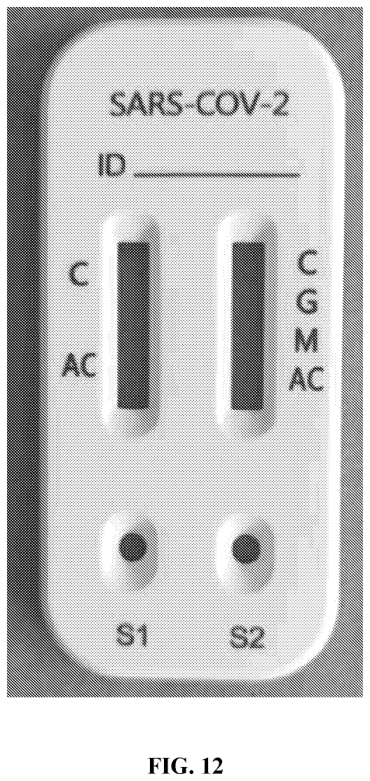

In some embodiments, the lateral flow device comprises 1, 2 or more pluralities of lateral flow regions. In some embodiments, the 2 or more pluralities of lateral flow regions are parallel to each other. In some embodiments, the 2 or more pluralities of lateral flow regions comprise a plurality for use as a control and a plurality for use for testing. FIG. 12 illustrates a configuration involving two pluralities of lateral flow regions, where the plurality on the left is for use as a control and the plurality on the right is for use for testing. In some embodiments, the plurality for use as a control is essentially as shown in FIG. 1 except for omission of the test line comprising a plurality of pre-set immobilized anti-human IgM capture antibodies (170b) and the test line comprising a plurality of pre-set immobilized anti-human IgG capture antibodies (170c).

In some embodiments, the pluralities of lateral flow regions form test strips. In some embodiments, the lateral flow device comprises 1, 2 or more test strips. In some embodiments, the lateral flow device comprises a sample test strip. In some embodiments, the lateral flow device comprises a sample test strip and a control strip. In some of these embodiments, the control strip is a positive control strip. In other embodiments, the control strip is a negative control strip. In some embodiments, the lateral flow device comprises 1, 2 or more control strips, e.g., in addition to the sample test strip.

### Sample Pad:

The lateral flow device comprises a sample application zone (110) having a sample pad (130) and optionally a sample port (120).

In one embodiment, when the sample application zone includes a sample port (120) and a sample pad (130), the liquid sample is dispensed (or at least an aliquot of the liquid sample is dispensed) to the sample port (120). In one embodiment, when the sample application zone lacks a sample port (120) but includes a sample pad (130), the liquid sample is dispensed onto the sample pad (130). In either case, the liquid sample can be dispensed to the sample application zone with or without a buffer.

In one embodiment, the sample pad (130) is in fluid communication with a conjugate pad (140). The sample pad (130) comprises material that can absorb a liquid sample and facilitates lateral or capillary flow of the liquid sample towards the conjugate pad (140). In one embodiment, the sample pad (130) does not contain immobilized gold-conjugates or immobilized detector molecules (e.g., capture antibodies). In one embodiment, the sample pad (130) comprises blood separator material. Suitable materials to make the sample pad (130) include absorbent materials, for example nitrocellulose, polyethylene membrane, nylon membrane, PVDF membrane, or paper.

In some embodiments, the lateral flow device comprises a second sample application zone, e.g., which is part of a second plurality of lateral flow regions. The second sample application zone and its components may have any of the features described herein, in the preceding paragraphs or elsewhere, for a sample application zone and components thereof. In some embodiments, the 2 or more sample pads are parallel to each other and/or are part of parallel pluralities of lateral flow regions.

Conjugate Pad:

In some embodiments, the lateral flow device comprises a conjugate pad (140) that comprises at least one type of a detectably labeled conjugate (e.g., gold-conjugate) that specifically binds to an analyte (e.g., antibodies from the subject) in the liquid sample and/or specifically binds to detector molecules immobilized at the detection zone. The conjugates are pre-set in place on the conjugate pad, or within the matrix of the conjugate pad. In one embodiment, the conjugates in dried form are pre-set on the conjugate pad. In one embodiment, the material selected to construct the conjugate pad (140) is absorbent and permits the dried conjugates to be held in place (e.g., dried conjugates do not migrate) when the conjugate pad (140) is dry. In one embodiment, the material selected to construct the conjugate pad (140) can absorb a liquid sample (and analytes within the liquid sample) and facilitates lateral or capillary flow of the liquid sample (and analytes) towards the detection zone (150). Upon dispensing the liquid sample (or an aliquot thereof) to the sample application zone (110) via the sample port (120) or the conjugate pad (140), the liquid sample and the analytes contained therein moves from the sample application zone and through the conjugate pad (140) which releases the dried conjugates to permit lateral flow of the liquid sample, the analytes and the gold-conjugates towards the detection zone. During lateral flow, the analytes in the liquid sample mix with the conjugates present in the conjugate pad (140). In one embodiment, the material selected to construct the conjugate pad (140) permits selective binding between the analytes in the liquid sample and the conjugates.

The dried conjugates in the conjugate pad (140) comprise a mixture of detectably labeled S1 spike protein conjugates, and detectably labeled biotin conjugates. In some embodiments, the detectably labeled S1 spike protein conjugates are S1 spike protein-gold conjugates, and/or the detectably labeled biotin conjugates are biotin-gold conjugates.

In one embodiment, the S1 spike subunit comprises a SARS-CoV-2 virus polypeptide having the amino acid sequence of SEQ ID NO:2, or a fragment of the S1 spike subunit. In one embodiment, the S1 spike polypeptide or fragment thereof, which is conjugated to a detectable label (e.g., gold particle), binds a human ACE2 receptor polypeptide.

In one embodiment, the detectably labeled biotin conjugate (e.g., biotin-gold conjugate) can bind avidin, streptavidin, neutravidin, or derivatives of these compounds.

In one embodiment, where a gold-conjugate is used, the gold particle of the conjugate comprises colloidal gold including gold microspheres or gold nanospheres (e.g., colloidal gold particles). It is well known that gold nanoparticles have optical and electronic properties (e.g., surface plasmon) that are tunable by changing the size, shape, surface chemistry or aggregation state. For example, monodisperse gold nanoparticles approximately 30 nm in size will be detectable as a red or pink color. As the size of the gold nanoparticles increase (e.g., approximately 40 nm) they will be detectable as blue or purple. In one embodiment, the shape and size of the gold particles used to produce the gold-conjugates are selected to be detectable as one color, or as different colors, for color detection at the test lines (170a, 170b and 170c) and the control line (180). In one embodiment, the gold particles are conjugated to the S1 spike polypeptide (or fragment thereof) or the biotin, using well known methods, including passive adsorption or covalent conjugation. In one embodiment, the S1 spike polypeptide (or fragment thereof) are conjugated to a first type of gold nanoparticles, and the biotin are conjugated to a second type of gold nanoparticles, wherein the first and second type of gold nanoparticles have the same color or different colors.

Biotin passively conjugated to 40 nm gold nanoparticles are available from Expedeon (SKU 240-0200). Biotin covalently conjugated to 40 nm gold nanoparticles are available from Abcam (catalog No. ab186922).

In one embodiment, during lateral flow of the liquid sample (with antibody analytes) through the conjugate pad (140), the antibody analytes can bind the S1 spike-gold conjugates. In one embodiment, the antibody analytes may include non-neutralizing anti-S1 spike IgM and/or IgG antibodies that specifically bind the detectably labeled S1 spike polypeptide conjugate (e.g., S1 spike polypeptide-gold conjugate). In one embodiment, the antibody analytes may include neutralizing anti-S1 spike IgM and/or IgG antibodies that specifically bind the S1 spike polypeptide-gold conjugate.

In one embodiment, during lateral flow of the liquid sample (with antibody analytes) through the conjugate pad (140), the biotin-gold conjugates move towards the detection zone. In one embodiment, the antibody analytes in the liquid sample do not bind, or exhibit very low binding, to the detectably labeled biotin conjugate (e.g., biotin-gold conjugate).

In some embodiments, the lateral flow device comprises 1, 2 or more conjugate pads, which may have any of the features described herein for a conjugate pad. In some embodiments, the 2 or more conjugate pads are parallel to each other. In some embodiments, the 2 or more sample pads are parallel to each other and/or are part of parallel pluralities of lateral flow regions.

In some embodiments, the lateral flow device comprises a second sample conjugate pad, e.g., which is part of a second plurality of lateral flow regions. The second sample conjugate pad and its components may have any of the features described herein, in the preceding paragraphs or elsewhere, for a conjugate pad and components thereof.

### Embodiment of a Lateral Flow Device without a Conjugate Pad

In one embodiment, the lateral flow device lacks a conjugate pad (140) but includes a sample pad (130) and optionally includes a sample port (120). In this embodiment, the sample pad (130) is in fluid communication with the lateral flow membrane, and the sample port (120) if present is in fluid communication with the sample pad (130). The liquid sample can be dispensed to the sample pad (130), or the sample port (120) if present. In one embodiment, the liquid sample can be pre-mixed with one or more detectably labeled reagents, e.g., comprising the conjugates discussed above (e.g., detectably labeled S1 spike conjugate and/or detectably labeled biotin conjugate, such as S1 spike-gold conjugate and/or biotin-gold conjugate) and the mixture can be dispensed to the sample pad (130) or the sample port (120). In one embodiment, the liquid sample and the one or more detectably labeled conjugate reagents (e.g., gold conjugate reagents) can be dispensed to the sample pad (130) or sample port (120) separately and in any order. Such a lateral flow device can be provided in a kit with the detectably labeled reagents, e.g., comprising the conjugates discussed above (e.g., detectably labeled S1 spike conjugate and/or detectably labeled biotin conjugate, such as S1 spike-gold conjugate and/or biotin-gold conjugate).

In one embodiment, a first detectably labeled conjugate reagent comprises detectably labeled S1 spike conjugates. In one embodiment, a second detectably labeled conjugate reagent comprises detectably labeled biotin conjugates. In one embodiment, a third detectably labeled conjugate reagent comprises both detectably labeled S1 spike conjugates and detectably labeled biotin conjugates. In one embodiment, a first gold-conjugate reagent comprises S1 spike-gold conjugates. In one embodiment, a second gold-conjugate reagent comprises biotin-gold conjugates. In one embodiment, a third gold-conjugate reagent comprises both S1 spike-gold conjugates and biotin-gold conjugates.

In one embodiment, the liquid sample can be pre-mixed with the first and second reagent, or with the third reagent, and the resulting mixture can be dispensed to the sample pad (130) or the sample port (120). In one embodiment, the liquid sample and the first and second conjugate reagents can be dispensed to the sample pad (130) or sample port (120) separately and in any order. In one embodiment, the liquid sample and the third conjugate reagent can be dispensed to the sample pad (130) or sample port (120) separately and in any order.

In one embodiment, the first and third conjugate reagents comprise an S1 spike subunit comprising a SARS-CoV-2 virus polypeptide having the amino acid sequence of SEQ ID NO:2, or a fragment of the S1 spike subunit. In one embodiment, the S1 spike polypeptide or fragment thereof binds a human ACE2 receptor polypeptide and is optionally conjugated to a gold particle.

In one embodiment, the second and third conjugate reagents comprise biotin conjugates which can bind avidin, streptavidin, neutravidin, or derivatives of these compounds.

In one embodiment, the conjugates in the first, second and third conjugate reagents comprise gold particles that are colloidal gold including gold microspheres or gold nanospheres. In one embodiment, the shape and size of the gold particles used to produce the gold-conjugates are selected to be detectable as one color, or as different colors, for color detection at the test lines (170a, 170b, 170c and/or 170d) and the control lines (180 and/or 180b). In one embodiment, colloidal gold particles are selected to be detectable as red, pink, blue or purple. In one embodiment, the gold particles are conjugated to the S1 spike polypeptide (or fragment thereof) or the biotin, using well known methods, including passive adsorption or covalent conjugation.

Biotin passively conjugated to 40 nm gold nanoparticles are available from Expedeon (SKU 240-0200). Biotin covalently conjugated to 40 nm gold nanoparticles are available from Abcam (catalog No. ab186922).

### Detection Zone

The lateral flow device comprises a detection zone (150) which comprises a lateral flow membrane with a plurality of test lines (e.g., 170a, 170b and 170c) and a control line (180).

In one embodiment, the lateral flow membrane comprises an absorbent material which facilitates migration of the liquid sample (and analytes contained therein) in a lateral or capillary flow manner. The test lines and control line comprise immobilized capture reagents. In one embodiment, the immobilized capture reagents comprise ACE2 receptor polypeptides or anti-human antibodies in the form of an IgM or IgG type. In one embodiment, the anti-human IgM or IgG capture antibodies comprise monoclonal or polyclonal antibodies made in goat, rabbit, mouse, rat, or other mammal. In one embodiment, the anti-human IgM or IgG capture antibodies comprise un-conjugated antibodies. In one embodiment, the anti-human IgG capture antibodies comprise an IgG-Fc fragment. The anti-human IgM or IgG capture antibodies can be obtained from a commercial entity including Bethyl Laboratories (e.g., anti-human IgM from goat, catalog No. A80-100A; anti-human IgM antibody from rabbit, catalog No. A80-101A; anti-human IgG-Fc fragment antibody from goat, catalog No. A80-104A; or anti-human IgG-Fc fragment antibody from rabbit, catalog No. A80-105A). The anti-human IgM or IgG capture antibodies can be obtained from other commercial sources, including SeraCare, abcam, and Thermo Fisher. In one embodiment, the lateral flow membrane comprises an absorbent material which is suitable for binding between the immobilized capture reagents and antibody analytes contained in the liquid sample during the lateral flow.

The order of the test lines on the lateral flow membrane, 170a, 170b and 170c, can be arranged in any order. For example, the ordered arrangement of the test lines on the lateral flow membrane (oriented from the conjugate pad to the absorbent pad) can be (i) 170a, 170b and 170c; (ii) 170a, 170c and 170b; (iii) 170b, 170a and 170c; (iv) 170b, 170c and 170a; (v) 170c, 170a and 170b; or (vi) 170c, 170b and 170a. The descriptions of the test lines as first, second and third are merely exemplary.

In some embodiments, the lateral flow device comprises an second detection zone which comprises a lateral flow membrane with a test line and a control line. The second detection zone may be part of a second plurality of lateral flow regions. The second sample detection zone and its components may have any of the features described herein, in the preceding paragraphs or elsewhere, for a detection zone and components thereof. In some embodiments, the order of the test line 170d and the control line 180b on the lateral flow membrane can be arranged in any order. For example, the ordered arrangement of the test line and the control line can be (i) 170d and 180b; or (ii) 180b and 170d. The descriptions of the fourth test line and second control line are merely exemplary.

### A First Test Line:

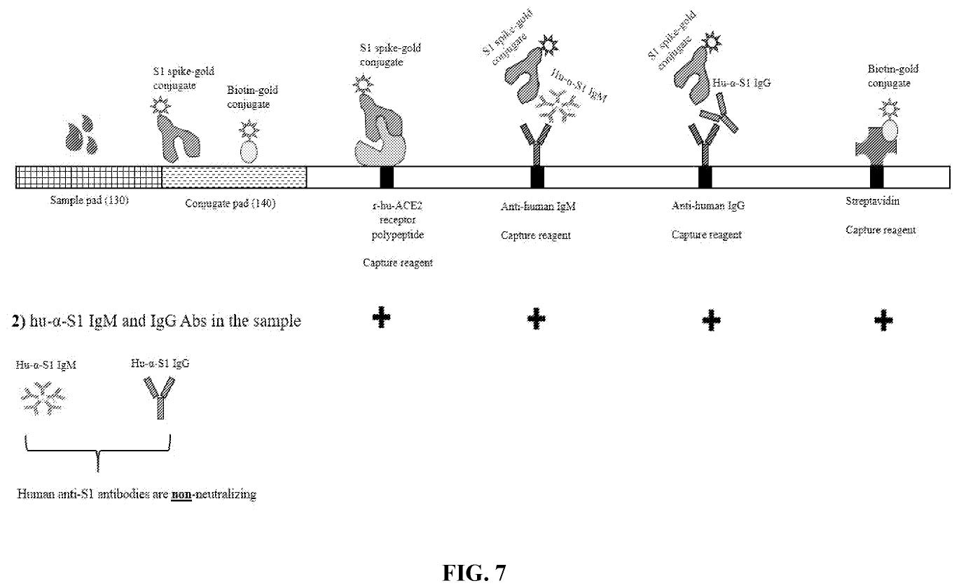

In one embodiment, the first test line (170a) comprises immobilized ACE2 receptor polypeptide (capture reagent), or a fragment of an ACE2 receptor polypeptide. In one embodiment, the immobilized ACE2 receptor capture polypeptide (or fragment thereof) binds an S1 spike subunit polypeptide, for example an S1 spike polypeptide-gold conjugate. In one embodiment, the immobilized ACE2 receptor capture polypeptide binds a receptor binding domain (RBD) on the S1 spike subunit polypeptide. In one embodiment, the immobilized ACE2 receptor capture polypeptide comprises a recombinant polypeptide. In one embodiment, the immobilized ACE2 receptor capture polypeptide comprises a human ACE2 receptor polypeptide, for example having the amino acid sequence according to SEQ ID NO:3 (FIG. 7, UniProtKB Q9BYF1). In one embodiment, the immobilized ACE2 receptor capture polypeptide exhibits ACE2 enzyme function and binds S1 spike polypeptide, or the immobilized ACE2 receptor capture polypeptide is mutated to abolish ACE2 enzymatic activity but still binds S1 spike polypeptide. For example, the ACE2 receptor polypeptide comprises mutations at H374N and H378N positions to abolish ACE2 enzymatic activity but the immobilized ACE2 receptor capture polypeptide still binds S1 spike polypeptide (e.g., the positions for H374N and H378N are underlined and bolded in FIG. 7). In one embodiment, the immobilized ACE2 receptor capture polypeptide binds the receptor binding domain (RBD) on an S1 spike polypeptide from a SARS-CoV-2 virus, where the S1 spike polypeptide comprises the amino acid sequence of SEQ ID NO:2 (GenBank MN908947.3).

In one embodiment, during lateral flow of the liquid sample (with non-neutralizing IgM and/or IgG antibody analytes) through the lateral flow membrane (160), the ACE2 receptor capture polypeptides immobilized at test line (107a) binds the migrating detectably labeled S1 spike polypeptide conjugates (e.g., S1 spike polypeptide-gold conjugates). In one embodiment, binding between the immobilized ACE2 receptor capture polypeptides and the detectably labeled S1 spike polypeptide conjugates (e.g., S1 spike polypeptide-gold conjugates) gives a detectable color change.

In one embodiment, during lateral flow of the liquid sample (with neutralizing IgM and/or IgG antibody analytes) through the lateral flow membrane (160), the ACE2 receptor capture polypeptides immobilized at test line (107a) is blocked from binding the migrating S1 spike polypeptide-gold conjugates because the neutralizing antibodies bind the receptor binding domain (or a site that overlaps with the RBD) on the detectably labeled S1 spike polypeptide conjugates (e.g., S1 spike polypeptide-gold conjugates). In one embodiment, the lack of binding between the immobilized ACE2 receptor capture polypeptides and the detectably labeled S1 spike polypeptide conjugates (e.g., S1 spike polypeptide-gold conjugates) gives no detectable color change.

### A Second Test Line:

In one embodiment, the second test line (170b) comprises immobilized anti-human IgM capture antibody. In one embodiment, the immobilized anti-human IgM capture antibody binds neutralizing and/or non-neutralizing human anti-S1 IgM antibody analyte that may be present in the lateral flowing liquid sample. In one embodiment, during lateral flow of the liquid sample through the lateral flow membrane (160), the human anti-S1 IgM antibody analyte is bound to the detectably labeled S1 spike polypeptide conjugate (e.g., S1 spike polypeptide-gold conjugate) S1 spike polypeptide-gold conjugate forming an analyte-S1 conjugate complex, wherein the complex can bind the immobilized anti-human IgM capture antibody. In one embodiment, binding between the immobilized anti-human IgM capture antibody and the complex give a detectable color change.

### A Third Test Line:

In one embodiment, the third test line (170c) comprises immobilized anti-human IgG capture antibody. In one embodiment, the immobilized anti-human IgG capture antibody binds neutralizing and/or non-neutralizing human anti-S1 IgG antibody analyte that may be present in the lateral flowing liquid sample. In one embodiment, during lateral flow of the liquid sample through the lateral flow membrane (160), the human anti-S1 IgG antibody analyte is bound to the detectably labeled S1 spike polypeptide conjugate (e.g., S1 spike polypeptide-gold conjugate) forming an analyte-S1-gold conjugate complex, wherein the complex can bind the immobilized anti-human IgG capture antibody. In one embodiment, binding between the immobilized anti-human IgG capture antibody and the complex give a detectable color change.

### A Fourth Test Line:

In some embodiments, a fourth test line is present in a second plurality of lateral flow regions, e.g., that serves as a control. The fourth test line and components thereof may have any of the features described herein for a first test line and components thereof, e.g., in the paragraph above regarding a first test line or elsewhere. In one embodiment, the fourth test line comprises immobilized ACE2 receptor polypeptide (capture reagent), or a fragment of an ACE2 receptor polypeptide.

In one embodiment, the immobilized ACE2 receptor capture polypeptide (or fragment thereof) binds an S1 spike subunit polypeptide, for example an S1 spike polypeptide-gold conjugate. In one embodiment, the immobilized ACE2 receptor capture polypeptide binds a receptor binding domain (RBD) on the S1 spike subunit polypeptide. In one embodiment, the immobilized ACE2 receptor capture polypeptide comprises a recombinant polypeptide. In one embodiment, the immobilized ACE2 receptor capture polypeptide comprises a human ACE2 receptor polypeptide, for example having the amino acid sequence according to SEQ ID NO:3 (FIG. 7, UniProtKB Q9BYF1). In one embodiment, the immobilized ACE2 receptor capture polypeptide exhibits ACE2 enzyme function and binds S1 spike polypeptide, or the immobilized ACE2 receptor capture polypeptide is mutated to abolish ACE2 enzymatic activity but still binds S1 spike polypeptide. For example, the ACE2 receptor polypeptide comprises mutations at H374N and H378N positions to abolish ACE2 enzymatic activity but the immobilized ACE2 receptor capture polypeptide still binds S1 spike polypeptide (e.g., the positions for H374N and H378N are underlined and bolded in FIG. 7). In one embodiment, the immobilized ACE2 receptor capture polypeptide binds the receptor binding domain (RBD) on an S1 spike polypeptide from a SARS-CoV-2 virus, where the S1 spike polypeptide comprises the amino acid sequence of SEQ ID NO:2 (GenBank MN908947.3).

In one embodiment, binding between the immobilized ACE2 receptor capture polypeptides and the detectably labeled S1 spike polypeptide conjugates (e.g., S1 spike polypeptide-gold conjugates) gives a detectable color change.

In one embodiment, the lack of binding between the immobilized ACE2 receptor capture polypeptides and the detectably labeled S1 spike polypeptide conjugates (e.g., S1 spike polypeptide-gold conjugates) gives no detectable color change.

### A Control Line:

In one embodiment, the control line (180) comprises an immobilized capture reagent that binds biotin. In one embodiment, the immobilized capture reagent comprises avidin polypeptide or a derivative of an avidin polypeptide, including glycosylated or non-glycosylated forms, recombinant or native forms, or full-length or truncated forms. In one embodiment, the immobilized capture reagents comprise avidin, streptavidin, NEUTRAVIDIN, EXTRAVIDIN, CAPTAVIDIN, or NEUTRALITE AVIDIN. In one embodiment, the immobilized capture reagents comprise avidin polypeptide or a derivative of an avidin polypeptide that binds the detectably labeled biotin conjugate (e.g., biotin-gold conjugate) present in the lateral flow. In one embodiment, during lateral flow of the liquid sample through the lateral flow membrane (160), the detectably labeled biotin conjugate (e.g., biotin-gold conjugate) can bind the immobilized avidin capture reagents. In one embodiment, binding between the immobilized avidin capture reagents and the detectably labeled biotin conjugate (e.g., biotin-gold conjugate) gives a detectable color change.

In some embodiments, the lateral flow device comprises 1, 2 or more control lines. Where a second control line is present, it may, e.g., be part of a second detection zone in a second plurality of lateral flow regions. The second control line and components thereof may have any of the features described herein for a control line and components thereof, e.g., in the preceding paragraph or elsewhere.

### Absorbent Pad:

The lateral flow device comprises an absorbent pad (190) that is in fluid communication with the lateral flow membrane (160). The absorbent pad (190) comprises material that can absorb a liquid sample and facilitates lateral or capillary flow of the liquid sample (and analytes contained therein), gold-conjugates and buffer, from the lateral flow membrane (160). In one embodiment, the absorbent pad (190) does not contain pre-set gold-conjugates or immobilized detector molecules (e.g., capture antibodies). In one embodiment, the absorbent pad (190) absorbs excess liquid, excess gold-conjugates, and excess analytes, that did not react/bind with the capture antibodies or immobilized streptavidin. In one embodiment, lateral flow of the liquid sample and analytes contained therein through the absorbent pad (190) does not produce a detectable color change. In one embodiment, the absorbent pad (190) can draw the liquids, analytes and gold-conjugates through the length of the lateral flow device. Suitable materials to make the absorbent pad (190) include absorbent materials, for example nitrocellulose, nitrocellulose blends with cellulose or polyester, polyethylene membrane, nylon membrane, PVDF membrane, or paper (e.g., untreated or porous paper), rayon, glass fiber, acrylonitrile copolymer, plastic, glass or nylon. In some embodiments, the lateral flow device comprises 1, 2 or more absorbent pads. Where a second absorbent pad is present, it may, e.g., be part of a second plurality of lateral flow regions. The second absorbent pad and components thereof may have any of the features described herein for an absorbent pad and components thereof, e.g., in the preceding paragraph or elsewhere.

### Support:

The lateral flow device may comprise a planar support (200) affixed on one side to a single type or different types of absorbent material(s) to form multiple lateral flow regions of the lateral flow device. In one embodiment, the one or both sides of the material used to make the support (200) is substantially impervious to fluids. In one embodiment, the material selected to construct the support (200) has minimal interference with lateral flow of the absorbent material(s) affixed thereon that make up the multiple lateral flow regions.

### Housing:

The lateral flow device may comprise a housing (not shown in FIG. 1 or 2). The housing can be an enclosure for the absorbent materials comprising lateral flow regions. In one embodiment, the housing includes a base and a lid with end walls and side walls. In one embodiment, the lid can include one or more cut-out regions for liquid sample dispensing (e.g., at the position of the sample port (120) or sample pad (130)) and an observation window (e.g., for observing the detection zone (150)). In one embodiment, the housing can be detachable to permit access to the lateral flow regions, or the housing can be non-detachable. In one embodiment, the housing is made of material that is substantially impervious to liquid and can be molded into a desired shape and thickness. In one embodiment, the housing comprises plastic. In some embodiments, e.g., where a second plurality of lateral flow regions is present, the housing comprises a set of cut-out regions, e.g., including a cut-out region for liquid sample dispensing (e.g., at the position of the sample port or sample pad of the second plurality of lateral flow regions) and an observation window, e.g., for observing the detection zone of the second plurality of lateral flow regions.

### Test Results:

The lateral flow device includes a detection zone (150) which contains four test lines (see FIGS. 1 and 2, 170a, 170b, 170c) and a control line (180). Test line 1 (170a) contains immobilized human ACE2 receptor polypeptide (e.g., recombinant human ACE2 receptor polypeptide). Test line 2 (170b) contains immobilized anti-human IgM antibody. Test line 3 (170c) contains immobilized anti-human IgG antibody. Control line (180) contains immobilized streptavidin.

The detection zone (150) can give three different results depending on the presence or absence of circulating anti-S1 spike antibodies in the liquid sample of the subject being tested.

In one embodiment, a liquid sample from a subject who lacks any circulating anti-S1 spike antibodies (or who has a circulating antibody titer that is too low to detect) can give the following test results: positive (test line 1 (170a)); negative (test line 2 (170b)); negative (test line 3 (170c)); and positive (control line (180)).

In one embodiment, a liquid sample from a subject who has circulating anti-S1 spike antibodies but lacks neutralizing antibodies can give the following test results: positive (test line 1 (170a)); positive (test line 2 (170b)); positive (test line 3 (170c)); and positive (control line (180)).

In one embodiment, a liquid sample from a subject who has circulating anti-S1 spike antibodies which include at least some neutralizing antibodies can give the following test results: negative (test line 1 (170a)); positive (test line 2 (170b)); positive (test line 3 (170c)); and positive (control line (180)).

In one embodiment, a test result is deemed invalid when a liquid sample from a subject is dispensed on the lateral flow device and gives a negative result on the control line (180) which indicates that the lateral flow device used in this instance did not work properly.

In one embodiment, a test result is deemed invalid when a liquid sample (e.g., without neutralizing IgM and/or IgG antibody analytes) is dispensed on the application zone of a negative control strip and gives no detectable color change on the test line that comprises immobilized ACE2 receptor polypeptide (capture reagents), or a fragment of an ACE2 receptor polypeptide.

What is claimed:

1. A lateral flow device comprising: a plurality of lateral flow regions arranged in the order:
a) a sample application zone (110) for dispensing a liquid sample thereupon wherein the sample application zone (110) comprises an absorbent material;
b) a conjugate pad (140) comprising an absorbent material and a plurality of pre-set gold-conjugates which include (i) a plurality of coronavirus S1 spike polypeptide-gold conjugates and (ii) a plurality of biotin-gold conjugates;
c) a detection zone (150) comprising a lateral flow membrane (160) comprising an absorbent material which includes a plurality of test lines and a control line,
wherein the plurality of test lines includes (i) a test line (170a) comprising a plurality of pre-set immobilized human ACE2 receptor polypeptide capture reagents, (ii) a test line (170b) comprising a plurality of pre-set immobilized anti-human IgM capture antibodies, (iii) a test line (170c) comprising a plurality of pre-set immobilized anti-human IgG capture antibodies, and the control line (180) comprises a plurality of pre-set immobilized capture reagents that bind biotin; and
d) an absorbent pad (190) comprising a second absorbent material,
wherein the lateral flow membrane (160) is in fluid communication with the absorbent pad (190) and wherein (i) the sample application zone (110) is in fluid communication with the conjugate pad (140) and the conjugate pad (140) is in fluid communication with the lateral flow membrane (160); or (ii) the sample pad (130) is in fluid communication with the lateral flow membrane (160).

2. A kit comprising a detectably labeled S1 spike conjugate, a detectably labeled biotin conjugate, and a lateral flow device, wherein the lateral flow device comprises a plurality of lateral flow regions arranged in the order:
a) a sample application zone (110) for dispensing a liquid sample thereupon wherein the sample application zone (110) comprises an absorbent material;
b) at least one of (i) a conjugate pad (140) comprising an absorbent material and a plurality of pre-set detectably labeled conjugates which include (i) a plurality of detectably labeled coronavirus S1 spike polypeptide conjugates and (ii) a plurality of detectably labeled biotin conjugates or (ii) a sample pad (130);
c) a detection zone (150) comprising a lateral flow membrane (160) comprising an absorbent material which includes a plurality of test lines and a control line,
wherein the plurality of test lines includes (i) a test line (170a) comprising a plurality of pre-set immobilized human ACE2 receptor polypeptide capture reagents, (ii) a test line (170b) comprising a plurality of pre-set immobilized anti-human IgM capture antibodies, (iii) a test line (170c) comprising a plurality of pre-set immobilized anti-human IgG capture antibodies, and the control line (180) comprises a plurality of pre-set immobilized capture reagents that bind biotin; and
d) an absorbent pad (190) comprising a second absorbent material,
wherein the lateral flow membrane (160) is in fluid communication with the absorbent pad (190) and wherein (i) the sample application zone (110) is in fluid communication with the conjugate pad (140) and the conjugate pad (140) is in fluid communication with the lateral flow membrane (160); or (ii) the sample pad (130) is in fluid communication with the lateral flow membrane (160).

3. The lateral flow device of claim 1 or the kit of claim 2, wherein the detectably labeled coronavirus S1 spike polypeptide conjugates comprise coronavirus S1 spike polypeptide gold-conjugates.

4. The lateral flow device or kit of any one of the preceding claims, wherein the detectably labeled biotin conjugates comprise biotin gold-conjugates.

5. The lateral flow device or kit of any one of the preceding claims, wherein the test line (170a) comprising the plurality of pre-set immobilized human ACE2 receptor polypeptide capture reagents is the first test line, the test line (170b) comprising the plurality of pre-set immobilized anti-human IgM capture antibodies is the second test line, and the test line (170c) comprising the plurality of pre-set immobilized anti-human IgG capture antibodies is the third test line, in order of increasing distance from the sample application zone.

6. The lateral flow device or kit of any one of the preceding claims, wherein the sample application zone (110) comprises a sample pad (130) and an optional sample port (120), wherein the sample port (120) if present is in fluid communication with the sample pad (130).

7. The lateral flow device or kit of any one of the preceding claims, wherein the coronavirus S1 spike polypeptide in the plurality of detectably labeled coronavirus S1 spike polypeptide conjugates comprises S1 spike polypeptide from SARS-CoV-2 and having the amino acid sequence of SEQ ID NO:2.

8. The lateral flow device or kit of any one of the preceding claims, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents at the test line (170a).

9. The lateral flow device or kit of any one of the preceding claims, wherein the coronavirus S1 spike polypeptide in the plurality of detectably labeled coronavirus S1 spike polypeptide conjugates comprises a receptor binding domain (RBD) that binds a human anti-S1 neutralizing IgM antibody, or the receptor binding domain (RBD) binds a human anti-S1 neutralizing IgG antibody.

10. The lateral flow device or kit of any one of the preceding claims, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents, and the receptor binding domain (RBD) binds a human anti-S1 neutralizing IgM antibody.

11. The lateral flow device or kit of claim 10, wherein binding between the receptor binding domain (RBD) of the coronavirus S1 spike polypeptide and the human anti-S1 neutralizing IgM antibody blocks binding between the receptor binding domain (RBD) of the coronavirus S1 spike polypeptide and the pre-set immobilized human ACE2 receptor polypeptide capture reagents at the test line (170a).

12. The lateral flow device or kit of any one of the preceding claims, wherein the coronavirus S1 spike polypeptide in the plurality of detectably labeled coronavirus S1 spike polypeptide conjugates comprises a receptor binding domain (RBD) that binds an epitope on the pre-set immobilized human ACE2 receptor polypeptide capture reagents, and the receptor binding domain (RBD) binds a human anti-S1 neutralizing IgG antibody.

13. The lateral flow device or kit of claim 12, wherein binding between the receptor binding domain (RBD) of the coronavirus S1 spike polypeptide and the human anti-S1 neutralizing IgG antibody blocks binding between the receptor binding domain (RBD) of the coronavirus S1 spike polypeptide and the pre-set immobilized human ACE2 receptor polypeptide capture reagents at the test line (170a).

14. The lateral flow device or kit of any one of the preceding claims, wherein the coronavirus S1 spike polypeptide in the plurality of detectably labeled coronavirus S1 spike polypeptide conjugates comprises non-receptor binding domains that bind a human anti-S1 non-neutralizing IgM antibody.

15. The lateral flow device or kit of claim 14, wherein binding between the non-receptor binding domain of the coronavirus S1 spike polypeptide and the human anti-S1 non-neutralizing IgM antibody does not block binding between the receptor binding domain (RBD) of the coronavirus S1 spike polypeptide and the pre-set immobilized human ACE2 receptor polypeptide capture reagents at the test line (170a).

16. The lateral flow device or kit of any one of the preceding claims, wherein the coronavirus S1 spike polypeptide in the plurality of detectably labeled coronavirus S1 spike polypeptide conjugates comprises non-receptor binding domains that bind a human anti-S1 non-neutralizing IgG antibody.

17. The lateral flow device or kit of claim 16, wherein binding between the non-receptor binding domain of the coronavirus S1 spike polypeptide and the human anti-S1 non-neutralizing IgG antibody does not block binding between the receptor binding domain (RBD) of the coronavirus S1 spike polypeptide and the pre-set immobilized human ACE2 receptor polypeptide capture reagents at the test line (170a).

18. The lateral flow device or kit of any one of the preceding claims, wherein the plurality of detectably labeled coronavirus S1 spike polypeptide conjugates comprise gold nanoparticles conjugated to coronavirus S1 spike polypeptides, and wherein the gold nanoparticles have a detectable color.

19. The lateral flow device or kit of any one of the preceding claims, wherein the plurality of detectably labeled biotin conjugates comprise gold nanoparticles conjugated to biotin, and wherein the gold nanoparticles have a detectable color.

20. The lateral flow device or kit of any one of the preceding claims, wherein the pre-set immobilized human ACE2 receptor polypeptide capture reagents in the test line (170a) comprise an epitope that binds a receptor binding domain (RBD) of an S1 spike polypeptide from SARS-CoV-2, wherein the pre-set immobilized human ACE2 receptor polypeptide comprises the amino acid sequence of SEQ ID NO:3, or a portion thereof.

21. The lateral flow device or kit of any one of the preceding claims, wherein the pre-set immobilized anti-human IgM capture antibodies in the test line (170b) bind a human anti-S1 IgM antibody (e.g., neutralizing and non-neutralizing antibodies).

22. The lateral flow device or kit of claim 21, wherein the pre-set immobilized anti-human IgM capture antibodies in the test line (170b) bind a plurality of a complex comprising the human anti-S1 IgM antibody bound to the detectably labeled coronavirus S1 spike polypeptide conjugate.

23. The lateral flow device or kit of any one of the preceding claims, wherein the pre-set immobilized anti-human IgG capture antibodies in the third test line (170b) bind a human anti-S1 IgG antibody (e.g., neutralizing and non-neutralizing antibodies).

24. The lateral flow device or kit of claim 23, wherein the pre-set immobilized anti-human IgG capture antibodies in the third test line (170c) bind a plurality of a complex comprising the human anti-S1 IgG antibody bound to the detectably labeled coronavirus S1 spike polypeptide conjugate.

25. The lateral flow device or kit of any one of the preceding claims, wherein the pre-set immobilized capture reagents that bind biotin at the control line (180) bind a plurality of detectably labeled biotin conjugates.

26. The lateral flow device or kit of any one of the preceding claims, which is disposed in a housing which includes a base, a lid, two end walls and two side walls.

27. The lateral flow device or kit of claim 26, wherein the lid of the housing includes a first cut-out region at the position of the sample port (120) or the sample pad (130) for liquid sample dispensing, and the lid includes a second cut-out region at the detection zone (150) for use as an observation window.

28. The lateral flow device or kit of any one of the preceding claims, wherein the capture reagents that bind biotin comprise avidin, streptavidin, NEUTRAVIDIN, EXTRAVIDIN, CAPTAVIDIN, or NEUTRALITE AVIDIN, or a truncated form thereof that retains biotin-binding activity, optionally wherein the avidin, streptavidin, NEUTRAVIDIN, EXTRAVIDIN, CAPTAVIDIN, or NEUTRALITE AVIDIN is glycosylated.

29. The lateral flow device or kit of any one of the preceding claims, wherein the lateral flow device further comprises a second plurality of lateral flow regions arranged in the order:
2a) a second sample application zone for dispensing a liquid sample thereupon wherein the sample application zone comprises an absorbent material;
2b) at least one of (i) a second conjugate pad comprising an absorbent material and a plurality of pre-set detectably labeled conjugates which include (1) a plurality of detectably labeled coronavirus S1 spike polypeptide conjugates and (2) a plurality of detectably labeled biotin conjugates, or (ii) a second sample pad;
2c) a second detection zone comprising a lateral flow membrane comprising an absorbent material which includes a test line comprising a plurality of pre-set immobilized human ACE2 receptor polypeptide capture reagents and a control line comprising a plurality of pre-set immobilized capture reagents that bind biotin; and
2d) a second absorbent pad comprising an absorbent material,
wherein the lateral flow membrane is in fluid communication with the absorbent pad, and
wherein (i) the sample application zone is in fluid communication with the second conjugate pad and the second conjugate pad is in fluid communication with the lateral flow membrane of the second detection zone, or (ii) the second sample pad (130) is in fluid communication with the lateral flow membrane of the second detection zone.

30. The kit of any one of claims 2-29, wherein the lateral flow device further comprises a second plurality of lateral flow regions arranged in the order:
2a) a second sample application zone for dispensing a liquid sample thereupon wherein the sample application zone comprises an absorbent material;
2b) at least one of (i) a second conjugate pad comprising an absorbent material and a plurality of pre-set detectably labeled conjugates which include (1) a plurality of detectably labeled coronavirus S1 spike polypeptide conjugates and (2) a plurality of detectably labeled biotin conjugates, or (ii) a second sample pad;
2c) a second detection zone comprising a lateral flow membrane comprising an absorbent material which includes a test line comprising a plurality of pre-set immobilized human ACE2 receptor polypeptide capture reagents and a control line comprising a plurality of pre-set immobilized capture reagents that bind biotin; and
2d) a second absorbent pad comprising an absorbent material,
wherein the lateral flow membrane is in fluid communication with the absorbent pad, and
wherein (i) the sample application zone is in fluid communication with the second conjugate pad and the second conjugate pad is in fluid communication with the lateral flow membrane of the second detection zone, or (ii) the second sample pad (130) is in fluid communication with the lateral flow membrane of the second detection zone.

31. The lateral flow device or kit of claim 29 or the kit of claim 30, wherein the second sample application zone comprises the second sample pad and an optional sample port, wherein the optional sample port if present is in fluid communication with the second sample pad.

32. The lateral flow device or kit of any one of claims 29-31, wherein the detectably labeled coronavirus S1 spike polypeptide conjugates in the second conjugate pad comprise S1 spike polypeptide from SARS-CoV-2 and having the amino acid sequence of SEQ ID NO:2.

33. The lateral flow device or kit of any one of claims 29-32, wherein the detectably labeled coronavirus S1 spike polypeptide conjugates in the second conjugate pad comprise a receptor binding domain (RBD) that binds an epitope on the pre-set immobilized human ACE2 receptor polypeptide capture reagents at the test line (170d).

34. The lateral flow device or kit of any one of claims 29-33, wherein the detectably labeled coronavirus S1 spike polypeptide conjugates in the second conjugate pad comprise a receptor binding domain (RBD) that binds a human anti-S1 neutralizing IgM antibody, or the receptor binding domain (RBD) binds a human anti-S1 neutralizing IgG antibody.

35. The lateral flow device or kit of any one of claims 29-34, wherein the detectably labeled coronavirus S1 spike polypeptide conjugates in the second conjugate pad comprise a receptor binding domain (RBD) that binds an epitope on the pre-set immobilized human ACE2 receptor polypeptide capture reagents at the test line (170d), and the receptor binding domain (RBD) binds a human anti-S1 neutralizing IgM antibody.

36. The lateral flow device or kit of any one of claims 29-35, wherein the coronavirus S1 spike polypeptide in the plurality of detectably labeled coronavirus S1 spike polypeptide conjugates of the second plurality of lateral flow regions comprises a receptor binding domain (RBD) that binds an epitope on the pre-set immobilized human ACE2 receptor polypeptide capture reagents at the test line (170d), and the receptor binding domain (RBD) binds a human anti-S1 neutralizing IgG antibody.

37. The lateral flow device or kit of any one of claims 29-36, wherein the detectably labeled coronavirus S1 spike polypeptide conjugates in the second conjugate pad comprise non-receptor binding domains that bind a human anti-S1 non-neutralizing IgM antibody.

38. The lateral flow device or kit of claim 37, wherein binding between the non-receptor binding domain of the coronavirus S1 spike polypeptide and the human anti-S1 non-neutralizing IgM antibody does not block binding between the receptor binding domain (RBD) of the coronavirus S1 spike polypeptide and the pre-set immobilized human ACE2 receptor polypeptide capture reagents at the test line.

39. The lateral flow device or kit of any one of claims 29-38, wherein the detectably labeled coronavirus S1 spike polypeptide conjugates in the second conjugate pad comprise non-receptor binding domains that bind a human anti-S1 non-neutralizing IgG antibody.

40. The lateral flow device or kit of any one of claims 29-39, wherein the detectably labeled coronavirus S1 spike polypeptide conjugates comprise coronavirus S1 spike polypeptide gold-conjugates.

41. The lateral flow device or kit of any one of claims 29-40, wherein the detectably labeled biotin conjugates in the second conjugate pad comprise biotin-gold conjugates.

42. The lateral flow device or kit of the immediately preceding claim, wherein the biotin-gold conjugates comprise gold nanoparticles conjugated to biotin, and wherein the gold nanoparticles have a detectable color.

43. The lateral flow device or kit of any one of claims 29-42, wherein the pre-set immobilized human ACE2 receptor polypeptide capture reagents in the test line of the second detection zone comprise an epitope that binds a receptor binding domain (RBD) of an S1 spike polypeptide from SARS-CoV-2, wherein the pre-set immobilized human ACE2 receptor polypeptide comprises the amino acid sequence of SEQ ID NO:3, or a portion thereof.

44. The lateral flow device or kit of any one of claims 29-43, which is disposed in a housing which includes a base, a lid, two end walls and two side walls, wherein the lid of the housing includes a third cut-out region at the position of the sample port or the sample pad for liquid sample dispensing, and the lid includes a fourth cut-out region at the detection zone for use as an observation window,
wherein the third and fourth cut-out regions are positioned over the second plurality of lateral flow regions.

45. A method for detecting in a liquid sample from a subject the presence or absence of anti-S1 antibody analytes of IgM and/or IgG type, the method comprising the steps of:
a) providing a liquid sample from the subject, wherein the liquid sample is suspected of containing human anti-S1 non-neutralizing antibody analytes of IgM and/or IgG type, and wherein the liquid sample is suspected of containing human anti-S1 neutralizing antibody analytes of IgM and/or IgG type;
b) dispensing the liquid sample onto the sample application zone (110) of a lateral flow device of any one of claims 1-44 (i) under a condition that is suitable for lateral flow of the liquid sample and the human anti-S1 non-neutralizing and neutralizing antibody analytes that may be contained therein, wherein the lateral flow moves the liquid sample or aliquot and the human anti-S1 non-neutralizing and neutralizing antibody analytes, if present, from the sample application zone (110) through the conjugate pad (140) through the detection zone (150) and through the absorbent pad (190), and (ii) under a condition that is suitable for lateral flow of the pre-set detectably labeled coronavirus S1 spike polypeptide conjugates and the pre-set detectably labeled biotin conjugates from the conjugate pad (140) through the detection zone (150) and through the absorbent pad (190), and (iii) under a condition that is suitable for binding of flowing detectably labeled coronavirus S1 spike polypeptide conjugates to the human anti-S1 non-neutralizing antibody analytes (if present) to form at least one S1 polypeptide-non-neutralizing antibody complex, and suitable for binding of flowing detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex, and (iv) under a condition that is suitable for binding flowing detectably labeled coronavirus S1 spike polypeptide conjugates to the pre-set immobilized human ACE2 receptor polypeptide capture reagents, and is suitable for binding the detectably labeled S1 polypeptide-non-neutralizing antibody complex to the pre-set immobilized anti-human IgM and IgG capture antibodies, and is suitable for binding the S1 polypeptide-neutralizing antibody complex to the pre-set immobilized anti-human IgM and IgG capture antibodies, and is suitable for binding flowing detectably labeled biotin conjugates to the pre-set immobilized capture reagents that bind biotin;
c) waiting a sufficient time for a color change to develop at the test line (170a), the test line (170b), the test line (170c) and the control line (180), or permitting a color change to develop at one or more test and/or control lines; and
d) detecting the color change at the test line (170a), the test line (170b), the test line (170c) and/or the control line (180).

46. A method for detecting in a liquid sample from a subject the presence or absence of anti-S1 antibody analytes of IgM and/or IgG type, the method comprising the steps of:
a) providing a liquid sample from the subject, wherein the liquid sample is suspected of containing human anti-S1 non-neutralizing antibody analytes of IgM and/or IgG type, and wherein the liquid sample is suspected of containing human anti-S1 neutralizing antibody analytes of IgM and/or IgG type, and mixing the liquid sample with a detectably labeled S1 spike conjugate and a detectably labeled biotin conjugate;
b) dispensing the liquid sample onto the sample application pad (130) or the sample port (120) of a lateral flow device of the kit of any one of claims 2-44 (i) under a condition that is suitable for lateral flow of the liquid sample and the human anti-S1 non-neutralizing and neutralizing antibody analytes that may be contained therein, wherein the lateral flow moves the liquid sample or aliquot and the human anti-S1 non-neutralizing and neutralizing antibody analytes, if present, from the sample application pad (130) or the sample port (120) through the detection zone (150) and through the absorbent pad (190), and (ii) under a condition that is suitable for lateral flow of the detectably labeled coronavirus S1 spike polypeptide conjugates and the detectably labeled biotin conjugates from the conjugate pad (140) through the detection zone (150) and through the absorbent pad (190), and (iii) under a condition that is suitable for binding of the detectably labeled coronavirus S1 spike polypeptide conjugates to the human anti-S1 non-neutralizing antibody analytes (if present) to form at least one S1 polypeptide-non-neutralizing antibody complex, and suitable for binding of the detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex, and (iv) under a condition that is suitable for binding flowing detectably labeled coronavirus S1 spike polypeptide conjugates to the pre-set immobilized human ACE2 receptor polypeptide capture reagents, and is suitable for binding the detectably labeled S1 polypeptide-non-neutralizing antibody complex to the pre-set immobilized anti-human IgM and IgG capture antibodies, and is suitable for binding the S1 polypeptide-neutralizing antibody complex to the pre-set immobilized anti-human IgM and IgG capture antibodies, and is suitable for binding flowing detectably labeled biotin conjugates to the pre-set immobilized capture reagents that bind biotin;
c) waiting a sufficient time for a color change to develop at the test line (170a), the test line (170b), the test line (170c) and the control line (180), or permitting a color change to develop at one or more test and/or control lines; and
d) detecting the color change at the test line (170a), the test line (170b), the test line (170c) and/or the control line (180).

47. A method for detecting in a liquid sample from a subject the presence or absence of anti-S1 antibody analytes of IgM and/or IgG type, the method comprising the steps of:
a) providing a first liquid sample from the subject and a second liquid sample of known composition, wherein the first liquid sample is suspected of containing human anti-S1 non-neutralizing antibody analytes of IgM and/or IgG type, and wherein the first liquid sample is suspected of containing human anti-S1 neutralizing antibody analytes of IgM and/or IgG type;
b) dispensing the first liquid sample onto the sample application zone and dispensing the second liquid sample onto the second sample application zone of the lateral flow device of any one of claims 29-44 (i) under a condition that is suitable for lateral flow of the liquid samples, wherein the lateral flow moves the first liquid sample from the sample application zone through the conjugate pad, through the detection zone, and through the absorbent pad, and moves the second liquid sample from the second sample application zone through the second conjugate pad, through the second detection zone, and through the second absorbent pad, and (ii) under a condition that is suitable for lateral flow of the pre-set detectably labeled coronavirus S1 spike polypeptide conjugates and the pre-set detectably labeled biotin conjugates from the conjugate pad and second conjugate pad through the detection zone and second detection zone and through the absorbent pad and second absorbent pad, and (iii) under a condition that is suitable for binding of flowing detectably labeled coronavirus S1 spike polypeptide conjugates to the human anti-S1 non-neutralizing antibody analytes (if present) to form at least one S1 polypeptide-non-neutralizing antibody complex, and suitable for binding of flowing detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex and that is suitable for binding flowing detectably labeled coronavirus S1 spike polypeptide conjugates to the pre-set immobilized human ACE2 receptor polypeptide capture reagents at the test line of the second detection zone, and is suitable for binding flowing detectably labeled biotin conjugates to the pre-set immobilized capture reagents that bind biotin at the control line of the second detection zone;
c) waiting a sufficient time for a color change to develop at the test lines and the control lines, or permitting a color change to develop at one or more test and/or control lines; and
d) detecting the color change at one or more of the test lines and/or the control lines.

48. A method for detecting in a liquid sample from a subject the presence or absence of anti-S1 antibody analytes of IgM and/or IgG type, the method comprising the steps of:
a) providing a first liquid sample from the subject and a second liquid sample of known composition, wherein the first liquid sample is suspected of containing human anti-S1 non-neutralizing antibody analytes of IgM and/or IgG type, and wherein the first liquid sample is suspected of containing human anti-S1 neutralizing antibody analytes of IgM and/or IgG type;
b) mixing the first liquid sample with a detectably labeled S1 spike conjugate and a detectably labeled biotin conjugate, and mixing the second liquid sample with a detectably labeled S1 spike conjugate and a detectably labeled biotin conjugate;
c) dispensing the first liquid sample onto the sample pad or sample port and dispensing the second liquid sample onto the second sample pad or second sample port of the lateral flow device of the kit of any one of claims 30-44 (i) under a condition that is suitable for lateral flow of the liquid samples, wherein the lateral flow moves the first liquid sample from the sample application zone through the detection zone, and through the absorbent pad, and moves the second liquid sample from the second sample application zone through the second detection zone, and through the second absorbent pad, and (ii) under a condition that is suitable for lateral flow of the detectably labeled coronavirus S1 spike polypeptide conjugates and the detectably labeled biotin conjugates through the detection zone and second detection zone and through the absorbent pad and second absorbent pad, and (iii) under a condition that is suitable for binding of the detectably labeled coronavirus S1 spike polypeptide conjugates to the human anti-S1 non-neutralizing antibody analytes (if present) to form at least one S1 polypeptide-non-neutralizing antibody complex, and suitable for binding of the detectably labeled coronavirus S1 spike polypeptide conjugates to the human anti-S1 neutralizing antibody analytes (if present) wherein the RBD of the S1 polypeptide binds to the neutralizing antibody to form at least one S1 polypeptide-neutralizing antibody complex and that is suitable for binding flowing detectably labeled coronavirus S1 spike polypeptide conjugates to the pre-set immobilized human ACE2 receptor polypeptide capture reagents at the test line of the second detection zone, and is suitable for binding flowing detectably labeled biotin conjugates to the pre-set immobilized capture reagents that bind biotin at the control line of the second detection zone;
d) waiting a sufficient time for a color change to develop at the test lines and the control lines, or permitting a color change to develop at one or more test and/or control lines; and
e) detecting the color change at one or more of the test lines and/or the control lines.

49. The method of claim 47 or 48, wherein the detecting the color change at the test line (170b) comprising the plurality of pre-set immobilized anti-human IgM capture antibodies indicates the liquid sample contains human anti-S1 spike IgM antibodies and the subject has an active SARS-Cov-2 infection and is likely to be contagious.

50. The method of any one of claims 47-49, wherein the detecting the color change at the test line (170c) comprising the plurality of pre-set immobilized anti-human IgG capture antibodies indicates the liquid sample contains human anti-S1 spike IgG antibodies and the subject has been infected with SARS-CoV-2 in the past but may not currently be contagious.

51. The method of any one of the claims 47-50, wherein the detecting the color change at the test line (170a) comprising the plurality of pre-set immobilized human ACE2 receptor polypeptide capture reagents indicates the liquid sample does not contain detectable human anti-S1 spike IgM neutralizing antibodies and does not contain detectable human anti-S1 spike IgG neutralizing antibodies.

52. The method of any one of claims 47-51, wherein a lack of a color change at the test line (170a) comprising the plurality of pre-set immobilized human ACE2 receptor polypeptide capture reagents indicates the liquid sample contains detectable human anti-S1 spike IgM neutralizing antibodies and/or the liquid sample contains detectable human anti-S1 spike IgG neutralizing antibodies.
